SEQUENCE LISTING (61 sequences)

SEQ ID NO: 1 - DNA, 1340 bp, Homo sapiens
  CDS (130)..(639)
ccattggccggcgtccccgccccagcgaacccggccccgcccccgaggcgccccattggccccgccgcgcgaaggcagagccgcggacgcccgggagcgacgagcgcgcagcgaaccgggtgccgggtcatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgaggccctgagaggccggcggctccccaagccattggccggcgtccccgccccagcgaacccggccccgcccccgaggcgccccattggccccgccgcgcgaaggcagagccgcggacgcccgggagcgacgagcgcgcagcgaaccgggtgccgggtcatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgaggccctgagaggccggcggctccccaag

SEQ ID NO: 2 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 3 - DNA, 878 bp, Homo sapiens
  CDS (1)..(417)
tacccgcatctggagggcgacgtgcgctggcgccgcctcttctcctccactcactttttcctgcgtgtggaccttggtggtcgggtgcaggggacgcgttggcggcacggccaggacagtatagtggagatccgttctgtccgtgtgggcactgtggtgatcaaagctgtgtactcaggcttctatgtggccatgaatcgcaggggccgcctctatgggtcgcgggtctactctgtggactgtaggttccgggagcgcatcgaggagaacggctacaacacatacgcctcgcgacgttggaggcaccgcggccgacccatgttcctggcacttgacagccaaggcattcccaggcaaggcagacggacacgacggcaccaactgtccacacacttcctgccagtcttggtctcgtcttgaagggcctgccaatggttcaggaggcatgggcggcacacagggcctggaagatccggagctgaacaaccaagggccaggccagagaccctgggccaacacgagtctttatgtcacaagccgggcgcccgctggctgccgggcatggagacatggcagggtccctgcaagtgaagccagcgctcagggggatacacagaactggcagtttgcatccatctagtttggagatgagaacactctgggcacagcacggagaggtttggaggtgggaacacacaccaggtggatgaggaaagcaggcagggaggaccggggagggtggacatgtcacggagggcagggcccgcgtcagtggggacagagacatgttcgcccatgtggccgaagcttggggctggagttaagagcacttcttgctcttccaaggggcctgagtttaattcttccacatcaggctg

SEQ ID NO: 4 - protein, 139 aa, Homo sapiens
YPHLEGDVRWRRLFSSTHFFLRVDLGGRVQGTRWRHGQDSIVEIRSVRVGTVVIKAVYSGFYVAMNRRGRLYGSRVYSVDCRFRERIEENGYNTYASRRWRHRGRPMFLALDSQGIPRQGRRTRRHQLSTHFLPVLVSS

SEQ ID NO: 5 - DNA, 510 bp, Homo sapiens
  CDS (1)..(510)
atgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 6 - DNA, 444 bp, Homo sapiens
  CDS (1)..(444)
accccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 7 - protein, 148 aa, Homo sapiens
TPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 8 - DNA, 550 bp, Homo sapiens
cgaaccgggtgccgggtcatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgaggccctgagaggccggcgg

SEQ ID NO: 9 - DNA, 456 bp, Homo sapiens
agatctaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctccctcgag

SEQ ID NO: 10 - DNA, 527 bp, Homo sapiens
agatctccaccatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctccctcgag

SEQ ID NO: 11 - DNA, 538 bp, Homo sapiens
caccgagctccccaccatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctaggtcgacggc

SEQ ID NO: 12 - DNA, 521 bp, Homo sapiens
  CDS (12)..(521)
agatctccaccatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggctacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 13 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGYNTYASQRWRRRGQPMFLALDRRGGPRPGGRTRRYHLSAHFLPVLVS

SEQ ID NO: 14 - DNA, 463 bp, Homo sapiens
  CDS (11)..(454)
caccagatctaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggctacaacacctacgcctcacagcgctggcgccgccgcggccagcccatgttcctggcgctggacaggaggggggggccccggccaggcggccggacgcggcggtaccacctgtccgcccacttcctgcccgtcctggtctccctcgagggc

SEQ ID NO: 15 - protein, 148 aa, Homo sapiens
TPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGYNTYASQRWRRRGQPMFLALDRRGGPRPGGRTRRYHLSAHFLPVLVS

SEQ ID NO: 16 - DNA, 444 bp, Homo sapiens
  CDS (1)..(444)
accccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactccaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 17 - protein, 148 aa, Homo sapiens
TPSASRGPRSYPHLEGDVRWRRLFSSTHFFLRVDPGGRVQGTRWRHGQDSILEIRSVHVGVVVIKAVSSGFYVAMNRRGRLYGSRLYTVDSRFRERIEENGHNTYASQRWRRRGQPMFLALDRRGGPRPGGRTRRYHLSAHFLPVLVS

SEQ ID NO: 18 - DNA, 444 bp, Homo sapiens
  CDS (1)..(444)
accccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactccaggttccgggagcgcatcgaagagaacggctacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 19 - protein, 148 aa, Homo sapiens
TPSASRGPRSYPHLEGDVRWRRLFSSTHFFLRVDPGGRVQGTRWRHGQDSILEIRSVHVGVVVIKAVSSGFYVAMNRRGRLYGSRLYTVDSRFRERIEENGYNTYASQRWRRRGQPMFLALDRRGGPRPGGRTRRYHLSAHFLPVLVS

SEQ ID NO: 20 - DNA, 462 bp, Homo sapiens
  CDS (4)..(450)
catatgactccttcagcttcccgtggtccgcgttcatatcctcacttagaaggcgatgtccgttggcgccgtttgttttcctctacccattttttcctgcgcgttgaccctggtggtcgtgtacaaggcactcgttggcgtcacggtcaagattcaattttagaaattcgtagtgttcatgttggtgttgtagttattaaagctgtttcttccggattttatgtcgcaatgaatcgtcgtggtcgtctgtatggttcccgtttatataccgttgattgtcgtttccgtgaacgtattgaagaaaatggacataatacttatgcatcacaacgttggcgtcgtcgtggtcaacctatgtttcttgctttagatcgtcgtggtggtcctcgtccgggtggtcgtactcgtcgttatcatttatctgcacacttccttccggttcttgtctcgtaataactcgag

SEQ ID NO: 21 - protein, 149 aa, Homo sapiens
MTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 22 - DNA, 465 bp, Homo sapiens
gcccatatgaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctagctcgagggc

SEQ ID NO: 23 - DNA, 466 bp, Homo sapiens
  CDS (8)..(454)
cagtcatatgaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggctacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgactcgagggc

SEQ ID NO: 24 - protein, 149 aa, Homo sapiens
MTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGYNTYASQRWRRRGQPMFLALDRRGGPRPGGRTRRYHLSAHFLPVLVS

SEQ ID NO: 25 - DNA, 466 bp, Homo sapiens
  CDS (8)..(454)
cagtcatatgaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactccaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgactcgagggc

SEQ ID NO: 26 - protein, 149 aa, Homo sapiens
MTPSASRGPRSYPHLEGDVRWRRLFSSTHFFLRVDPGGRVQGTRWRHGQDSILEIRSVHVGVVVIKAVSSGFYVAMNRRGRLYGSRLYTVDSRFRERIEENGHNTYASQRWRRRGQPMFLALDRRGGPRPGGRTRRYHLSAHFLPVLVS

SEQ ID NO: 27 - DNA, 466 bp, Homo sapiens
  CDS (8)..(454)
cagtcatatgaccccgagcgcgtcgcggggaccgcgcagctacccgcacctggagggcgacgtgcgctggcggcgtctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactccaggttccgggagcgcatcgaagagaacggctacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgactcgagggc

SEQ ID NO: 28 - protein, 149 aa, Homo sapiens
MTPSASRGPRSYPHLEGDVRWRRLFSSTHFFLRVDPGGRVQGTRWRHGQDSILEIRSVHVGVVVIKAVSSGFYVAMNRRGRLYGSRLYTVDSRFRERIEENGYNTYASQRWRRRGQPMFLALDRRGGPRPGGRTRRYHLSAHFLPVLVS

SEQ ID NO: 29 - DNA, 510 bp, Homo sapiens
  CDS (1)..(510)
atgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcaaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcc

SEQ ID NO: 30 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIKENGHNTYASQRWRRRGQPMFLALDRRGGPRPGGRTRRYHLSAHFLPVLVS

SEQ ID NO: 31 - DNA, 38 bp, Artificial Sequence
  Description of Artifical Sequence: Primer/Probe
agatctccaccatgcgccgccgcctgtggctgggcctg

SEQ ID NO: 32 - DNA, 44 bp, Artificial Sequence
  Description of Artifical Sequence: Primer/Probe
ctcgtcagatctccaccatgcgccgccgcctgtggctgggcctg

SEQ ID NO: 33 - DNA, 36 bp, Artificial Sequence
  Description of Artifical Sequence: Primer/Probe
ctcgagggagaccaggacgggcaggaagtgggcgga

SEQ ID NO: 34 - DNA, 42 bp, Artificial Sequence
  Description of Artifical Sequence: Primer/Probe
ctcgtcctcgagggagaccaggacgggcaggaagtgggcgga

SEQ ID NO: 35 - DNA, 30 bp, Artificial Sequence
  Description of Artifical Sequence: Primer/Probe
agatctaccccgagcgcgtcgcggggaccg

SEQ ID NO: 36 - DNA, 42 bp, Artificial Sequence; identical to SEQ ID NO: 34
  Description of Artifical Sequence: Primer/Probe

SEQ ID NO: 37 - DNA, 30 bp, Artificial Sequence
  Description of Artifical Sequence: Primer/Probe
ctcgtcctcgagggtaagcctatccctaac

SEQ ID NO: 38 - DNA, 31 bp, Artificial Sequence
  Description of Artifical Sequence: Primer/Probe
ctcgtcgggcccctgatcagcgggtttaaac

SEQ ID NO: 39 - DNA, 20 bp, Artificial Sequence
  Description of Artifical Sequence: Primer/Probe
cgtggtcatcaaagcagtgt

SEQ ID NO: 40 - DNA, 25 bp, Artificial Sequence
  Description of Artifical Sequence: Primer/Probe
ctcaggcttctacgtggccatgaac

SEQ ID NO: 41 - DNA, 20 bp, Artificial Sequence
  Description of Artifical Sequence: Primer/Probe
tgcagtccacggtgtagagt

SEQ ID NO: 42 - DNA, 21 bp, Artificial Sequence
  Description of Artifical Sequence: Primer/Probe
tggagatccgctctgtacacg

SEQ ID NO: 43 - DNA, 27 bp, Artificial Sequence
  Description of Artifical Sequence: Primer/Probe
cctgaggacactgctttgatgaccacg

SEQ ID NO: 44 - DNA, 19 bp, Artificial Sequence
  Description of Artifical Sequence: Primer/Probe
cggttcatggccacgtaga

SEQ ID NO: 45 - DNA, 20 bp, Artificial Sequence
  Description of Artifical Sequence: Primer/Probe
tggagatccgctctgtacac

SEQ ID NO: 46 - DNA, 26 bp, Artificial Sequence
  Description of Artifical Sequence: Primer/Probe
tcatcaaagcagtgtcctcaggcttc

SEQ ID NO: 47 - DNA, 20 bp, Artificial Sequence
  Description of Artifical Sequence: Primer/Probe
tgcagtccacggtgtagagt

SEQ ID NO: 48 - protein, 12 aa, Artificial Sequence
  Description of Artifical Sequence:  Synthetic peptide for use as immunogen
CPGGRTRRYHLS

SEQ ID NO: 49 - protein, 11 aa, Artificial Sequence
  Description of Artifical Sequence:  Synthetic peptide for use as immunogen
CSQRWRRRGQP

SEQ ID NO: 50 - protein, 16 aa, Artificial Sequence
  Description of Artifical Sequence:  Synthetic peptide for use as immunogen
CSTHFFLRVDPGGRVQ

SEQ ID NO: 51 - protein, 13 aa, Artificial Sequence
  Description of Artifical Sequence:  Synthetic peptide for use as immunogen
CRFRERIEENGHN

SEQ ID NO: 52 - protein, 13 aa, Artificial Sequence
  Description of Artifical Sequence:  Synthetic peptide for use as immunogen
CRPGGRTRRYHLS

SEQ ID NO: 53 - DNA, 23 bp, Artificial Sequence
  Description of Artifical Sequence: Primer/Probe
gtggaaaagaacggcagtaaata

SEQ ID NO: 54 - DNA, 26 bp, Artificial Sequence
  Description of Artifical Sequence: Primer/Probe
tgccctacctcaaggttctcaagcac

SEQ ID NO: 55 - DNA, 25 bp, Artificial Sequence
  Description of Artifical Sequence: Primer/Probe
acttctgcattggaactatttatcc

SEQ ID NO: 56 - DNA, 23 bp, Artificial Sequence
  Description of Artifical Sequence: Primer/Probe
cagtagttttccagcctttcttg

SEQ ID NO: 57 - DNA, 30 bp, Artificial Sequence
  Description of Artifical Sequence: Primer/Probe
tggactgttttctttcttctcaaaattttc

SEQ ID NO: 58 - DNA, 20 bp, Artificial Sequence
  Description of Artifical Sequence: Primer/Probe
tccagcagggagatttcttt

SEQ ID NO: 59 - DNA, 20 bp, Artificial Sequence
  Description of Artifical Sequence: Primer/Probe
gaggccaccaactcttcttc

SEQ ID NO: 60 - DNA, 25 bp, Artificial Sequence
  Description of Artifical Sequence: Primer/Probe
ctcctccttctcctctccttccagc

SEQ ID NO: 61 - DNA, 20 bp, Artificial Sequence
  Description of Artifical Sequence: Primer/Probe
gtgattgtagctccgcacat

(19) United States
(12) Patent Application Publication
(10) Pub. No.: US 2006/0141565 A1
(43) Pub. Date: Jun. 29, 2006

(54) Novel fibroblast growth factor and uses thereof

(76) Inventors: Meera Patturajan, Caldwell, NJ (US); Isabelle Millet, Milford, CT (US); Sampath Kumar, North Haven, CT (US); Charles Miller, Guilford, CT (US); Peter Mezes, Old Lyme, CT (US); Denise Lepley, East Granby, CT (US); Catherine Burgess, Wethersfield, CT (US); Richard Shimkets, Guilford, CT (US)

Correspondence Address: CURAGEN CORPORATION, 322 EAST MAIN STREET, BRANFORD, CT, 06405, US

(21) Appl. No.: 11/005,836

(22) Filed: Dec. 6, 2004

Related U.S. Application Data
(63) Continuation-in-part of application No. 10/384,974, filed on Mar. 10, 2003, now abandoned.
(63) Continuation-in-part of application No. 10/637,313, filed on Aug. 8, 2003.
Application No. 10/637,313 is a continuation-in-part of application No. 09/569,269, filed on May 11, 2000, now abandoned.
(60) Provisional application No. 60/134,315, filed on May 14, 1999.
(60) Provisional application No. 60/188,274, filed on Mar. 10, 2000.
(60) Provisional application No. 60/527,883, filed on Dec. 5, 2003.

(51) Int. Cl. C12P 21/06; C07H 21/04; C12N 1/21; C07K 14/50

(52) U.S. Cl. 435/69.1; 530/399; 435/320.1; 435/325; 435/252.33; 536/23.5

(57) ABSTRACT

The present invention generally relates to nucleic acids, proteins, and antibodies. The invention relates more particularly to nucleic acid molecules, proteins, and antibodies of Fibroblast Growth Factor-20 (FGF-22), or its fragments, derivatives, variants, homologs, analogs, or a combination thereof.

This application is a continuation-in-part of U.S. patent application Ser. Nos. 10/384,974, filed Mar. 10, 2003, and 10/637,313, filed Aug. 8, 2003, both of which are continuation-in-part of U.S. patent application Ser. No. 09/569,269, filed May 11, 2000, which claims priority of the U.S. Provisional Application Nos. 60/134,315, filed May 14, 1999, and 60/188,274, filed Mar. 10, 2000. This application also claims the priority benefits of U.S. Provisional Application No. 60/527,883, filed Dec. 5, 2003. The contents of each of these applications are incorporated herein by reference in their entireties.

### 1. FIELD OF THE INVENTION

The present invention generally relates to nucleic acids, proteins, antibodies and their uses. The invention relates more particularly to nucleic acid molecules, proteins, and antibodies of Fibroblast Growth Factor-22 (FGF-22), or its fragments, derivatives, variants, homologs, analogs, or a combination thereof, and their uses.

### 2. BACKGROUND OF THE INVENTION

The fibroblast growth factors (“FGFs”) are large family of growth factors that are highly conserved at both structural and amino acid levels. Previously described members of the FGF family regulate diverse cellular functions such as growth, survival, apoptosis, motility and differentiation (Szebenyi & Fallon (1999) Int. Rev. Cytol. 185, 45-106). These molecules transduce signals intracellularly via high affinity interactions with cell surface tyrosine kinase FGF receptors (FGFRs), four of which have been identified (Xu, X., Weinstein, M., Li, C. & Deng, C. (1999) Cell Tissue Res. 296, 33-43; Klint, P. & Claesson-Welsh, L. (1999) Front. Biosci. 4, 165-177). Alternative splicing in the extracellular domain results in receptor isolforms with different ligand binding specificities. These FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions, and to sequester FGFs and protect them from degradation (Szebenyi, G. & Fallon, J. F. (1999)). Due to its growth-promoting capabilities, one member of the FGF family, FGF-7, is currently in clinical trials for the treatment of chemotherapy-induced mucositis (Danilenko, D. M. (1999) Toxicol. Pathol. 27, 64-71).

In addition to participating in normal growth and development, known FGFs have also been implicated in the generation of pathological states, including cancer (Basilico, C & Moscatelli, D. (1992) Adv. Cancer Res. 59, 115-165). FGFs may contribute to malignancy by directly enhancing the growth of tumor cells. For example, autocrine growth stimulation through the co-expression of FGF and FGFR in the same cell leads to cellular transformation (Matsumoto-Yoshitomi, et al. (1997) Int. J. Cancer 71, 442-450). Likewise, the constitutive activation of FGFR via mutation or rearrangement leads to uncontrolled proliferation (Lorenzi, et al. (1996) Proc. Natl. Acad. Sci. USA. 93, 8956-8961; Li, et al. (1997) Oncogene 14, 1397-1406). Furthermore, some FGFs are angiogenic (Gerwins, et al. (2000) Crit. Rev. Oncol. Hematol. 34, 185-194). Such FGFs may contribute to the tumorigenic process by facilitating the development of the blood supply needed to sustain tumor growth. Not surprisingly, at least one FGF is currently under investigation as a potential target for cancer therapy (Gasparini (1999) Drugs 58, 17-38).

Expression of FGFs and their receptors in the brains of perinatal and adult mice has been examined. Messenger RNA all known FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrate similar spatiotemporal expression, suggesting that FGF-6 and FGFR-4 plays significant roles in the maturation of nervous system as a ligand-receptor system. According to Ozawa et al., these results strongly suggest that the various FGFs and their receptors are involved in the regulation of a variety of developmental processes of brain, such as proliferation and migration of neuronal progenitor cells, neuronal and glial differentiation, neurite extensions, and synapse formation.

Other members of the FGF polypeptide family include the FGF receptor tyrosine kinase (FGFRTK) family and the FGF receptor heparin sulfate proteoglycan (FGFRHS) family. These members interact to regulate active and specific FGFR signal transduction complexes. These regulatory activities are diversified throughout a broad range of organs and tissues, and in both normal and tumor tissues, in mammals. Regulated alternative messenger RNA (mRNA) splicing and combination of variant subdomains give rise to diversity of FGFRTK monomers. Divalent cations cooperate with the FGFRHS to conformationally restrict FGFRTK trans-phosphorylation, which causes depression of kinase activity and facilitates appropriate activation of the FGFR complex by FGF. For example, it is known that different point mutations in the FGFRTK commonly cause craniofacial and skeletal abnormalities of graded severity by graded increases in FGF-independent activity of total FGFR complexes. Other processes in which FGF family exerts important effects are liver growth and function and prostate tumor progression.

Glia-activating factor (GAF), another FGF family member, is a heparin-binding growth factor that was purified from the culture supernatant of a human glioma cell line. See, Miyamoto et al. 1993, Mol Cell Biol 13(7): 4251-4259. GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N terminus with respect to those predicted by the cDNA sequence, except the initiation methionine. The rat FGF 9 cDNA was also cloned, and the structural analysis indicated that the FGF-9 gene is highly conserved.

Citation or discussion of a reference herein shall not be construed as an admission that such is prior art to the present invention.

### 3. SUMMARY OF THE INVENTION

The present invention is based, in part, upon the discovery of a nucleic acid encoding a novel protein having homology to members of the Fibroblast Growth Factor (FGF) family of proteins. The present invention provides nucleic acids and proteins (including peptides and polypeptides) of FGF-22, its variants, derivatives, homologs, and analogs (collectively referred as “CG54455”). The present invention also provides antibodies against a CG54455 protein.

In one aspect, the invention provides an isolated CG54455 protein. In some embodiments, the isolated protein comprises the amino acid sequence of SEQ ID NO:2. In other embodiments, the invention includes a variant of SEQ ID NO:2, in which some amino acids residues, e.g., no more than 1%, 2%, 3%, 5%, 10% or 15% of the amino acid sequences of SEQ ID NO;2 are changed. In some embodiments, the isolated FGF-22 protein comprise the amino acid sequence of a mature form of an amino acid sequence given by SEQ ID NO:2, or a variant of a mature form of an amino acid sequence given by SEQ ID NO:2. In a preferred embodiment, a matured form of FGF-22 consisting amino acids 23-170 of SEQ ID NO:2 or 23-170 of a variant of SEQ ID NO:2. Preferably, no more than 1%, 2%, 3%, 5%, 10% or 15% of the amino acid sequences of SEQ ID NO;2 are changed in the variant of the mature form of the amino acid sequence.

In another aspect, the invention provides a fragment of an FGF-22 protein, including fragments of variant FGF-22 proteins, mature FGF-22 proteins, and variants of mature FGF-22 proteins, as well as FGF-22 proteins encoded by allelic variants and single nucleotide polymorphisms of FGF-22 nucleic acids. An example of an FGF-22 protein is a fragment that includes residues 2-170, 23-170, or 33-170 of FGF-22.

In another aspect, the invention includes an isolated CG54455 nucleic acid molecule. The CG54455 nucleic acid molecule can include a sequence encoding any of the FGF-22 proteins, variants, or fragments disclosed above, or a complement to any such nucleic acid sequence. In one embodiment, the sequences include those disclosed in SEQ ID NO:1, 3, 5, 6, 8, 9, 10, 11, 12, 14, 16, 18, 20, 22, 23, 25, or 27. In other embodiments, the FGF-22 nucleic acids include a sequence wherein nucleotides different from those given in SEQ ID NO:1 may be incorporated. Preferably, no more than 1%, 2%, 3,%, 5%, 10%, 15%, or 20% of the nucleotides are so changed.

In one embodiment, the nucleic acid encodes a protein fragment that includes residues 2-170, 23-170, or 33-170 of SEQ ID NO:2.

In other embodiments, the invention includes fragments or complements of these nucleic acid sequences. Vectors and cells incorporating CG54455 nucleic acids are also included in the invention. The present invention further provides methods of isolating a CG54455 protein by culturing the host cells containing a CG54455 nucleic acid in a suitable nutrient medium, and isolating one or more expressed CG54455 proteins. The host cells can be a prokaryotic cell or a eukaryotic cell. In a preferred embodiment, the host cell is E. coli. In another preferred embodiment, the host cell is a mammalian cell, such as a CHO cell.

In another embodiment, the present invention provides a method of stimulating proliferation, differentiation or migration of epithelial cells comprising administering to a subject in need thereof an effective amount of a composition comprising one or more CG54455 proteins or nucleic acids. In a specific embodiment, the epithelial cells are locate at the alimentary tract or pulmonary tract (e.g., trachea) of the subject.

The invention also includes antibodies that bind immunospecifically to any of the CG54455 proteins described herein. The CG54455 antibodies in various embodiments include, e.g., polyclonal antibodies, monoclonal antibodies, humanized antibodies and/or human antibodies.

The invention additionally provides pharmaceutical compositions that include a CG54455 protein, a CG54455 nucleic acid or a CG54455 antibody of the invention. Also included in the invention are kits that include, e.g., a CG54455 protein, a CG54455nucleic acid or a CG54455antibody.

Several methods are included in the invention. For example, a method is disclosed for determining the presence or amount of a CG54455 protein in a sample of animal or human serum or plasma. The method includes capturing CG54455 proteins with an immobilized monoclonal antibody to CG54455, addition of a rabbit secondary polyclonal antibody to CG54455 and detecting the rabbit antibody with donkey-anti-rabbit-horseradish peroxidase conjugate using standard ELISA techniques.

Similarly, the invention discloses a method for determining the presence or amount of a CG54455 nucleic acid molecule in a sample. The method includes contacting the sample with a probe that binds to the nucleic acid molecule; and determining the presence or amount of the probe bound to the nucleic acid molecule, such that the probe indicates the presence or amount of the CG54455 nucleic acid molecule in the sample.

Also provided by the invention is a method for identifying an agent that binds to a CG54455 protein. The method includes determining whether a candidate substance binds to a CG54455 protein. Binding of a candidate substance indicates the agent is an CG54455 protein binding agent.

The invention also includes a method for identifying a potential therapeutic agent for use in treatment of a pathology. The pathology is, e.g., related to aberrant expression, aberrant processing, or aberrant physiological interactions of a CG54455 protein of the invention. This method includes providing a cell which expresses the CG54455 protein and has a property or function ascribable to the protein; contacting the provided cell with a composition comprising a candidate substance; and determining whether the substance alters the property or function ascribable to the protein, in comparison to a control cell. Any such substance is identified as a potential therapeutic agent. Furthermore, therapeutic agents may be identified by subjecting any potential therapeutic agent identified in this way to additional tests to identify a therapeutic agent for use in treating the pathology.

In some embodiments, the property or function relates to cell growth or cell proliferation, and the substance binds to the protein, thereby modulating an activity of the protein. In one embodiment, the candidate substance has a molecular weight not more than about 1500 Da. In some embodiments, the candidate substance is an antibody. The invention additionally provides any therapeutic agent identified using a method such as those described herein.

The invention also includes a method for screening for a modulator of latency or predisposition to a disorder associated with aberrant expression, aberrant processing, or aberrant physiological interactions of a CG54455 protein. The method includes providing a test animal that recombinantly expresses the CG54455 protein of the invention and is at increased risk for the disorder; administering a test compound to the test animal; measuring an activity of the protein in the test animal after administering the compound; and comparing the activity of the FGF-22 protein in the test animal with the activity of the CG54455 protein in a control animal not administered the compound. If there is a change in the activity of the protein in the test animal relative to the control animal, the test compound is a modulator of latency of or predisposition to the disorder.

The invention also provides a method for determining the presence of or predisposition to a disease associated with altered levels of a CG54455 protein or of a CG54455 nucleic acid of the invention in a first mammalian subject. The method includes measuring the level of expression of the protein or the amount of the nucleic acid in a sample from the first mammalian subject; and comparing its amount in the sample to its amount present in a control sample from a second mammalian subject known not to have, or not to be predisposed to, the disease. An alteration in the expression level of the protein or the amount of the nucleic acid in the first subject as compared to the control sample indicates the presence of or predisposition to the disease.

Also provided by the invention is a method of treating a pathological state in a mammal, wherein the pathology is related to aberrant expression, aberrant processing, or aberrant physiological interactions of a CG54455 protein of the invention. The method includes administering to the mammal a protein of the invention in an amount that is sufficient to alleviate the pathological state, wherein the CG54455 protein is a protein having an amino acid sequence at least 85%, 90%, 95%, 96%, 97%, 98%, or even 99% identical to a protein comprising an amino acid sequence of SEQ ID NO:2, or a biologically active fragment thereof. In another related method, an antibody of the invention is administered to the mammal.

In another aspect, the invention, the invention includes a method of promoting growth of cells in a subject. The method includes administering to the subject a CG54455 protein of the invention in an amount and for a duration that are effective to promote cell growth. In some embodiments, the subject is a human, and the cells whose growth is to be promoted may be chosen from among cells in the vicinity of a wound, cells in the vascular system, cells involved in hematopoiesis, cells involved in erythropoiesis, cells in the lining of the gastrointestinal tract, and cells in hair follicles.

In a further aspect, the invention provides a method of inhibiting growth of cells in a subject, wherein the growth is related to expression of a CG54455 protein of the invention. This method includes administering to the subject a composition that inhibits growth of the cells. In a one embodiment, the composition includes an antibody of the invention. Significantly, the subject is a human, and the cells whose growth is to be inhibited are chosen from among transformed cells, hyperplastic cells, tumor cells, and neoplastic cells.

In a still further aspect, the invention provides a method of treating or preventing or delaying a tissue proliferation-associated disorder. The method includes administering to a subject in which such treatment or prevention or delay is desired a CG54455 antibody in an amount sufficient to treat, prevent, or delay a tissue proliferation-associated disorder in the subject.

The tissue proliferation-associated disorders diagnosed, treated, prevented or delayed using the CG54455 nucleic acid molecules, proteins or antibodies can involve epithelial cells, e.g., fibroblasts and keratinocytes in the anterior eye after surgery. Other tissue proliferation-associated disorders include, e.g., tumors, restenosis, psoriasis, Dupuytren's contracture, diabetic complications, Kaposi sarcoma, and rheumatoid arthritis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### 4. BRIEF DESCRIPTION OF THE DRAWINGS

FIG. 1 shows ClustalW alignment of human FGF-22 (CG54455-01) with other FGFs and FGF-22 from different species.

FIG. 2 shows Western Blot analysis of expression of HIS and V5 tagged FGF-22 in embryonic kidney 293 cells.

FIG. 3 shows Western Blot analysis of expression of HIS tagged mature form of FGF-22 (CG54455-06) in E. coli.

FIG. 4 shows CG54455-06-MSA fusion protein secreted by CHO-K cells.

FIG. 5 shows molecular modeling of FGF-22. (A) FGF-22 is modeled by FGF-2. Tyr103 of FGF-2 corresponds to His 124 of FGF-22; (B) Tyr and His are both planer hydrophobic residues.

FIG. 6 shows proliferation of Baf3-FGFR2 IIIb cells in the presence of CG54455-06.

FIG. 7 shows proliferation of Balb/MK cells (murine keratinocyte cell line) in the presence of CG54455-06.

FIG. 8 shows proliferation of CCD1106 cells (human keratinocyte cell line) in the presence of CG54455-06.

FIG. 9 shows proliferation of CCD1070sk cells (human fibroblast cell line) in the presence of CG54455-06.

FIG. 10 shows FGF22-MSA induces the proliferation of Balb-MK cells in a dose dependent fashion.

FIG. 11 shows proliferation results to recognize receptor specificity.

FIG. 12 shows receptor neutralization in the presence of the mature form of FGF-22.

FIG. 13 shows heparin sepharose affinity column chromatography (HSAC): E. coli lysate containing CG54455-14 was subjected to HSAC. (A) the elution profile of CG54455-14 on heparin sepharose column (HiTrap, 5 ml bed volume): step-elution profile with increment of 12.5% of elution buffer (25 mM sodium phosphate, pH 7.4, 3 M sodium chloride) up to 8 times. Fractions of 0.5 ml were collected, the letters at the bottom indicate the fraction number; (B) the fractions (20 μl) from the peaks (1 to 6) were subjected to SDS-PAGE followed by Western Blot analysis. CG54455 is eluted between 1.375-1.8 M NaCl (peaks 4, 5 and 6)

FIG. 14 shows CG54455-14 induces proliferation of BaF3R2 IIIb in a dose dependent fashion.

FIG. 15 shows sFGFR2 IIIb abrogates the Proliferative activity of CG54455-14 on BaF3R2b.

FIG. 16 shows CG54455-14 induces proliferation of Balb/MK cells in a dose dependent fashion.

FIG. 17 shows CG54455-14 stimulates p42/44 MAPK activation by protein phosphorylation. Lanes 1, 4, 7 and 10 are untreated Balb/MK cells; lanes 2, 5, 8 and 11 are Balb/MK cells treated with CG54455-14; and lanes 3, 6, 9 and 12 are Balb/MK cells treated with FGF-7.

FIG. 18 shows CG54455-14 stimulates p70S6 kinase. Lanes 1, 4, 7 and 10 are untreated Balb/MK cells; lanes 2, 5, 8 and 11 are Balb/MK cells treated with CG54455-14; and lanes 3, 6, 9 and 12 are Balb/MK cells treated with FGF-7.

FIG. 19 shows PD 98059 and LY294002 inhibit CG54455-14 induced Balb-MK proliferation.

FIG. 20 shows CG54455 expression profile in EpiDerm skin model, where expression is compared to FGF-10, FGF-7, and ADPR house keeping gene.

FIG. 21 shows CG54455 expression profile in human tissues, where expression is compared to FGF-10, FGF-7, and ADPR house keeping gene.

### 5. DETAILED DESCRIPTION OF THE INVENTION

This invention is based, in part, on the discovery of a class of proteins (including peptides and polypeptides) or nucleic acids encoding such proteins or their complementary strands, where the proteins comprise an amino acid sequence of SEQ ID NO:2 (170 amino acids), or its fragments, derivatives, variants, homologs, or analogs. This class of proteins and/or nucleic acid molecules is designated as “CG54455.” CG54455 was found to be expressed in skin and particular in the epidermis and can stimulate proliferation of epithelial cells but not mesenchymal cells in vitro, and thus have variety of uses, such as promoting wound healing and tissue repair, for generating skin graft substitute, and treating and/or preventing pathologies requiring such growth-like activity, e.g., mucositis, ulcer, stroke, disorders associated with radiation exposure.

For clarity of disclosure, and not by way of limitation, the detailed description of the invention is divided into the following subsections:
(i) CG54455
(ii) Methods of Preparing CG54455
(iii) Antibodies to CG54455
(iv) Structure Prediction and Functional Analysis of CG54455
(v) Uses of CG54455
(vi) Administration, Pharmaceutical Compositions and Kits

### 5.1. CG54455

The present invention provides nucleic acid molecules encoding FGF-22, or its fragments, derivatives, variants, homologs, or analogs, and the proteins (including peptides and polypeptides) encoded by such nucleic acid molecules. Such nucleic acid molecules and the proteins are collectively termed as “CG54455.” The present invention further provides antibodies against a CG54455 protein, and methods of use for CG54455 as well as antibodies against a CG54455 protein.

As used herein, the term “CG54455” refers to a class of proteins or nucleic acids encoding such proteins or their complementary strands, where the proteins comprise an amino acid sequence of SEQ ID NO:2 (170 amino acids), or its fragments, derivatives, variants, homologs, or analogs. In a preferred embodiment, a CG54455 protein retains at least some biological activity of FGF-22. As used herein, the term “biological activity” means that a CG54455 protein possesses some but not necessarily all the same properties of (and not necessarily to the same degree as) FGF-22.

A member (e.g., a protein and/or a nucleic acid encoding the protein) of the CG54455 family may further be given an identification name. For example, CG54455-01 (SEQ ID NOs:1 and 2) represents the first identified full length wild type FGF-22 (with sequences outside the coding region); CG54455-06 (SEQ ID NOs:9 and 7) represents a mature form of FGF-22 (having amino acids 23-170). Some members of the CG54455 family may differ in their nucleic acid sequences but encode the same CG54455 protein, e.g., CG54455-04 and CG54455-06 encode the same CG54455 protein. Table 1 shows a summary of some of the CG54455 family members. In one embodiment, the invention includes a variant of FGF-22 protein, in which some amino acids residues, e.g., no more than 1%, 2%, 3%, 5%, 10% or 15% of the amino acid sequence of FGF-22 (SEQ ID NO:2), are changed. In another embodiment, the invention includes nucleic acid molecules that can hybridize to FGF-22 under stringent hybridization conditions.

TABLE 1
Summary of some of the CG54455 family members

| | SEQ ID | |
|---|---|---|
| | NO | |
| | (DNA/ | |
| Name | Protein) | Brief Description |
| CG54455-01 | 1 and 2 | FGF-22 full length |
| CG54455-02 | 3 and 4 | Truncated form (amino acids 33-170*) with |
| | | various amino acids substitutions |
| CG54455-03 | 5 and 2 | FGF-22 full length |
| CG54455-04 | 6 and 7 | Mature form (amino acids 23-170) of FGF-22 |
| CG54455-05 | 8 and 2 | FGF-22 full length |
| CG54455-06 | 9 and 7 | Mature form (amino acids 23-170) of FGF-22 |
| CG54455-07 | 10 and 2 | FGF-22 full length |
| CG54455-08 | 11 and 2 | FGF-22 full length |
| CG54455-09 | 12 and 13 | Full length with ¹²⁴H→ Y (histidine is changed |
| | | to tyrosine at position 124) |
| CG54455-10 | 14 and 15 | Mature form (amino acids 23-170) |
| | | with ¹²⁴H→ Y |
| CG54455-11 | 16 and 17 | Mature form (amino acids 23-170) |
| | | with ¹¹³C→ S (cysteine changed to serine |
| | | at position 113) |
| CG54455-12 | 18 and 19 | Mature form (amino acids 23-170) |
| | | with ¹¹³C→ S and ¹²⁴H→ Y |
| CG54455-13 | 20 and 21 | Mature form (amino acids 1 and 23-170) |
| CG54455-14 | 22 and 21 | Mature form (amino acids 1, 23-170) |
| CG54455-15 | 23 and 24 | Mature form (amino acids 1, 23-170) |
| | | with ¹²⁴H→ Y |
| CG54455-16 | 25 and 26 | Mature form (amino acids 1, 23-170) |
| | | with ¹¹³C→ S |
| CG54455-17 | 27 and 28 | Mature form (amino acids 1, 23-170) |
| | | with ¹¹³C→ S and ¹²⁴H→ Y |
| CG54455-18 | 29 and 30 | Single nucleotide polymorphism |
| | | (SNP)13379002, where ³⁵⁸G→ A* (¹²⁰E→ K) |

*position of amino acid corresponds to SEQ ID NO: 2; and position of nucleic acid corresponds to SEQ ID NO: 5.

As used herein, the term “FGF-22” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “effective amount” refers to the amount of a therapy (e.g., a composition comprising a CG54455 protein) which is sufficient to reduce and/or ameliorate the severity and/or duration of a disease or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult that may induce alimentary mucositis (such as radiation, chemotherapy, or chemical warfare agents). In another embodiment, the subject is a farm animal (e.g., a horse, pig, or cow) or a pet (e.g., a dog or cat) that has been exposed to or is going to be exposed to a similar insult. The term “subject” is used interchangeably with “patient” in the present invention.

### 5.1.1. Identification of FGF-22

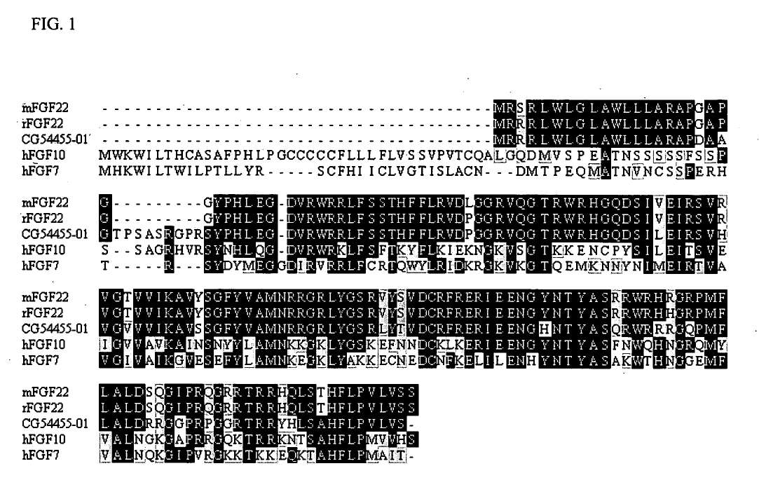

FGF-22 coding sequence was identified by homology mining after TBLASTN search GeneBank of genomic DNA sequence with FGF-10. The predicted open reading frame codes for a 170 amino acid long secreted protein with 54% identity to the Human Fibroblast Growth Factor 10 precursor (SWISSNEW-Acc. No. 015520), which is also known as Keratinocyte Growth Factor 2 (see PCT Publication No. WO 98/16642-A1). FGF-22 also bears 46% similarity to FGF-7, and conserved with rat and murine FGF-22. FIG.1 shows the ClustalW alignment of human FGF-22 (CG54455-01) with other FGFs.

FGF-22 protein sequence is predicted by the PSORT program to localize extracellularly with a certainty of 0.5374. The program SignalP predicts that there is a signal peptide, with the most likely cleavage site between residues 22 and 23 in the sequence AAG-TP.

Further analysis of the CG54455 proteins yield the following properties shown in Table 2.

TABLE 2
Protein Sequence Properties of CG54455

| | |
|---|---|
| SignalP analysis: | Cleavage site between residues 23 and 24 |
| PSORT II analysis: | |

The first 70 amino acids of 54455-01 (170 aa) were used for signal peptide
prediction

< Is the sequence a signal peptide?

| # Measure | | Position | Value | Cutoff | Conclusion |
|---|---|---|---|---|---|
| max. | C | 23 | 0.783 | 0.37 | YES |
| max. | Y | 23 | 0.781 | 0.34 | YES |
| max. | S | 10 | 0.984 | 0.88 | YES |
| mean | S | 1-22 | 0.887 | 0.48 | YES |

# Most likely cleavage site between pos. 22 and 23: AAG-TP
PSORT analysis

Prediction of Protein Translocation Sites version 5.8

| | |
|---|---|
| outside | Certainty = 0.5374(Affirmative) < succ> |
| microbody (peroxisome) | Certainty = 0.3298(Affirmative) < succ> |
| endoplasmic reticulum | Certainty = 0.1000(Affirmative) < succ> |
| (membrane) | |
| endoplasmic reticulum | Certainty = 0.1000(Affirmative) < succ> |
| (lumen) | |

PFam analysis predicts that FGF-22 protein contains the domain shown in Table 3.

TABLE 3

| Pfam | FGF-22 | Identities/Similarities | |
|---|---|---|---|
| Domain | Match Region | for the Matched Region | Expect Value |
| FGF | 17 . . . 146 | 63/147 (43%) | 1.8e−53 |
| | | 105/147 (71%) | |

FGF-22 also has high similarity to several segments from a human metalloprotease thrombospondin 1 (METH1) related EST (AC004449) of 38186 bp (see PCT publication WO 99/37660-A1). Metalloprotease thrombospondins are potent inhibitors of angiogenesis both in vitro and in vivo. Accordingly, FGF-22 nucleic acids and polypeptides may be useful in treating cancer and other disorders related to angiogenesis, including but not limited to, abnormal wound healing, inflammation, rheumatoid arthritis, psoriasis, endometrial bleeding disorders, diabetic retinopathy, some forms of macular degeneration, haemangiomas, and arterial-venous malformations.

### 5.1.2. FGF-22 Derivatives, Variants, Homologs, Analogs and Fragments

The present invention also provides derivatives, variants, homologs, analogs and fragments of FGF-22. For example, Section 6, infra, describes identification and cloning of additional FGF-22 variants.

A CG5445 protein described herein includes the product of a naturally occurring protein or precursor form or proprotein. The naturally occurring protein, precursor or proprotein includes, e.g., the full length gene product, encoded by the corresponding gene. The naturally occurring protein also includes the protein, precursor or proprotein encoded by an open reading frame described herein. A “mature” form of a protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor protein that has residues 1 to N, where residue 1 is the N-terminal methionine, may have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them. The program SignalP predicts that there is a signal peptide in FGF-22, with the most likely cleavage site between residues 22 and 23 in the sequence MG-TP. Accordingly, in one embodiment, a mature FGF-22 refers to a protein having amino acids 23-170 of the full length FGF-22.

In one embodiment, a CG54455 protein is a variant of FGF-22. It will be appreciated by those skilled in the art that DNA sequence polymorphisms that lead to changes in the amino acid sequences of the FGF-22 protein may exist within a population (e.g., the human population). Such genetic polymorphism in the FGF-22 gene may exist among individuals within a population due to natural allelic variation. Such natural allelic variations can typically result in 1-5% variance in the nucleotide sequence of the FGF-22 gene. Any and all such nucleotide variations and resulting amino acid polymorphisms in the FGF-22 protein, which are the result of natural allelic variation of the FGF-22 protein, are intended to be within the scope of the invention. A non-limiting example of a single nucleotide polymorphism (SNP) of CG54455 is SNP 13379002, where nucleotide G at position 358 of FGF-22 (SEQ ID NO:5) is changed to A, which results amino acid E at position 120 of FGF-22 (SEQ ID NO:2) is changed to K.

In another embodiment, CG54455 refers to a nucleic acid molecule encoding a FGF-22 protein from other species or the protein encoded thereby, and thus has a nucleotide or amino acid sequence that differs from the human sequence of FGF-22. Nucleic acid molecules corresponding to natural allelic variants and homologues of the FGF-22 cDNAs of the invention can be isolated based on their homology to the human FGF-22 nucleic acids disclosed herein using the human cDNAs, or a portion thereof, as a hybridization probe according to standard hybridization techniques under stringent hybridization conditions.

The invention also encompasses derivatives and analogs of FGF-22. The production and use of derivatives and analogs related to FGF-22 are within the scope of the present invention.

In a specific embodiment, the derivative or analog is functionally active, i.e., capable of exhibiting one or more functional activities associated with a wild-type FGF-22. Derivatives or analogs of FGF-22 can be tested for the desired activity by procedures known in the art, including but not limited to, using appropriate cell lines, animal models, and clinical trials.

In particular, FGF-22 derivatives can be made via altering FGF-22 sequences by substitutions, insertions or deletions that provide for functionally equivalent molecules. In one embodiment, such alteration of an FGF-22 sequence is done in a region that is not conserved in the FGF protein family. Due to the degeneracy of nucleotide coding sequences, other DNA sequences which encode substantially the same amino acid sequence as FGF-22 may be used in the practice of the present invention. These include, but are not limited to, nucleic acid sequences comprising all or portions of FGF-22 that are altered by the substitution of different codons that encode a functionally equivalent amino acid residue within the sequence, thus producing a silent change. Likewise, the FGF-22 derivatives of the invention include, but are not limited to, those containing, as a primary amino acid sequence, all or part of the amino acid sequence of FGF-22 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-22 derivatives of the invention also include, but are not limited to, those containing, as a primary amino acid sequence, all or part of the amino acid sequence of FGF-22 including altered sequences in which amino acid residues are substituted for residues with similar chemical properties. In a specific embodiment, 1, 2, 3, 4, or 5 amino acids are substituted.

Derivatives or analogs of FGF-22 include, but are not limited to, those proteins which are substantially homologous to FGF-22 or fragments thereof, or whose encoding nucleic acid is capable of hybridizing to the FGF-22 nucleic acid sequence.

The FGF-22 derivatives and analogs of the invention can be produced by various methods known in the art. The manipulations that result in their production can occur at the gene or protein level. For example, the cloned FGF-22 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-22, care should be taken to ensure that the modified gene remains within the same translational reading frame as FGF-22, uninterrupted by translational stop signals, in the gene region where the desired FGF-22 activity is encoded.

Additionally, the FGF-22-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the FGF-22 sequence may also be made at the protein level. Included within the scope of the invention are FGF-22 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2- groups, free COOH- groups, OH- groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-22 can be chemically synthesized. For example, a protein corresponding to a portion of FGF-22 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-22 sequence. Non-classical amino acids include, but are not limited to, the D-isomers of the common amino adds, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the FGF-22 derivative is a chimeric or fusion protein comprising FGF-22 or a fragment thereof fused via a peptide bond at its amino- and/or carboxy-terminus to a non-FGF-22 amino acid sequence. In one embodiment, the non-FGF-22 amino acid sequence is fused at the amino-terminus of an FGF-22 or a fragment thereof. In another embodiment, such a chimeric protein is produced by recombinant expression of a nucleic acid encoding the protein (comprising an FGF-22-coding sequence joined in-frame to a non-FGF-22 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-22 with a heterologous signal sequence is expressed such that the chimeric protein is expressed and processed by the cell to the mature FGF-22 protein. The primary sequence of FGF-22 and non-FGF-22 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-22 molecule fused to a heterologous (non-FGF-22) protein-encoding sequence may be constructed. In a specific embodiment, such chimeric construction can be used to enhance one or more desired properties of an FGF-22, including but not limited to, FGF-22 stability, solubility, or resistance to proteases. In another embodiment, chimeric construction can be used to target FGF-22 to a specific site. In yet another embodiment, chimeric construction can be used to identify or purify an FGF-22 of the invention, such as a His-tag, a FLAG tag, a green fluorescence protein (GFP), β-galactosidase, a maltose binding protein (MalE), a cellulose binding protein (CenA) or a mannose protein, etc.

In some embodiment, a CG54455 protein can be modified so that it has an extended half-life in vivo using any methods known in the art. For example, Fc fragment of human IgG or inert polymer molecules such as high molecular weight polyethyleneglycol (PEG) can be attached to a CG54455 protein. PEG can be attached to a CG54455 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG54455 protein. Unreacted PEG can be separated from CG54455-PEG conjugates by size-exclusion or by ion-exchange chromatography. PEG-derivatized conjugates can be tested for in vivo efficacy using methods known to those of skill in the art.

A CG54455 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG54455 refers to CG54455-01 (SEQ ID NOs:1 and 2), CG54455-02 (SEQ ID NOs:3 and 4), CG54455-03 (SEQ ID NOs:5 and 2), CG54455-04 (SEQ ID NOs:6 and 7), CG54455-05 (SEQ ID NOs:8 and 2), CG54455-06 (SEQ ID NOs:9 and 7), CG54455-07 (SEQ ID NOs:10 and 2), CG54455-08 (SEQ ID NOs:11 and 2), CG54455-09 (SEQ ID NOs:12 and 13), CG54455-10 (SEQ ID NOs:14 and 15), CG54455-11 (SEQ ID NOs:16 and 17), CG54455-12 (SEQ ID NOs:18 and 19), CG54455-13 (SEQ ID NOs:20 and 21), CG54455-14 (SEQ ID NOs:22 and 21), CG54455-15 (SEQ ID NOs:23 and 24), CG54455-16 (SEQ ID NOs:25 and 26), CG54455-17 (SEQ ID NOs:27 and 28), CG54455-18 (SEQ ID NOs: 29 and 30), or a combination thereof.

### 5.2. Methods of Preparing CG54455

Any techniques known in the art can be used in purifying a CG54455 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG54455 may be monitored by one or more in vitro or in vivo assays. The purity of CG54455 can be assayed by any methods known in the art, such as but not limited to, gel electrophoresis. See Scopes, supra. In some embodiment, the CG54455 proteins employed in a composition of the invention can be in the range of 80 to 100 percent of the total mg protein, or at least 80%, at least 85%, at least 90%, at least 95%, or at least 98% of the total mg protein. In one embodiment, one or more CG54455 proteins employed in a composition of the invention is at least 99% of the total protein. In another embodiment, CG54455 is purified to apparent homogeneity, as assayed, e.g., by sodium dodecyl sulfate polyacrylamide gel electrophoresis.

Methods known in the art can be utilized to recombinantly produce CG54455 proteins. A nucleic acid sequence encoding a CG54455 protein can be inserted into an expression vector for propagation and expression in host cells.

An expression construct, as used herein, refers to a nucleic acid sequence encoding a CG54455 protein operably associated with one or more regulatory regions that enable expression of a CG54455 protein in an appropriate host cell. “Operably-associated” refers to an association in which the regulatory regions and the CG54455 sequence to be expressed are joined and positioned in such a way as to permit transcription, and ultimately, translation.

The regulatory regions necessary for transcription of CG54455 can be provided by the expression vector. A translation initiation codon (ATG) may also be provided if a CG54455 gene sequence lacking its cognate initiation codon is to be expressed. In a compatible host-construct system, cellular transcriptional factors, such as RNA polymerase, will bind to the regulatory regions on the expression construct to effect transcription of the modified CG54455 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CMT sequence, and the like. The non-coding region 3′ to the coding sequence may contain transcriptional termination regulatory sequences, such as terminators and polyadenylation sites.

In order to attach DNA sequences with regulatory functions, such as promoters, to a CG54455 gene sequence or to insert a CG54455 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG54455 sequence operably associated with regulatory regions can be directly introduced into appropriate host cells for expression and production of a CG54455 protein without further cloning. See, e.g., U.S. Pat. No. 5,580,859. The expression constructs can also contain DNA sequences that facilitate integration of a CG54455 sequence into the genome of the host cell, e.g., via homologous recombination. In this instance, it is not necessary to employ an expression vector comprising a replication origin suitable for appropriate host cells in order to propagate and express CG54455 in the host cells.

A variety of expression vectors may be used, including but are not limited to, plasmids, cosmids, phage, phagemids or modified viruses. Such host-expression systems represent vehicles by which the coding sequences of a CG54455 gene may be produced and subsequently purified, but also represent cells which may, when transformed or transfected with the appropriate nucleotide coding sequences, express CG54455 in situ. These include, but are not limited to, microorganisms such as bacteria (e.g., E. coli and B. subtilis) transformed with recombinant bacteriophage DNA, plasmid DNA or cosmid DNA expression vectors containing CG54455 coding sequences; yeast (e.g., Saccharomyces, Pichia) transformed with recombinant yeast expression vectors containing CG54455 coding sequences; insect cell systems infected with recombinant virus expression vectors (e.g., baculovirus) containing CG54455 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG54455 coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG54455 molecule. For example, mammalian cells such as Chinese hamster ovary cells (CHO) can be used with a vector bearing promoter element from major intermediate early gene of cytomegalocirus for effective expression of a CG54455 sequence (Foecking et al., 1986, Gene 45:101; and Cockett et al., 1990, Bio/Technology 8:2).

In bacterial systems, a number of expression vectors may be advantageously selected depending upon the use intended for the CG54455 molecule being expressed. For example, when a large quantity of a CG54455 is to be produced, for the generation of pharmaceutical compositions of a CG54455 molecule, vectors that direct the expression of high levels of readily purified fusion protein products may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD protein. These recombinant proteins may be directed into periplasmic space for correct folding and maturation. The fused part can be used for affinity purification of the expressed protein. Presence of cleavage sites for specific proteases like enterokinase allows the CG54455 protein to be cleaved from the fusion protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG54455 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG54455 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG54455 in infected hosts (see, e.g., Logan & Shenk, 1984, Proc. Natl. Acad. Sci. USA 8 1:355-359). Specific initiation signals may also be required for efficient translation of inserted CG54455 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Billner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. Expression in a bacterial or yeast system can be used if post-translational modifications turn to be non-essential for a desired activity of CG54455. In a preferred embodiment, E. coli is used to express a CG54455 sequence.

For long-term, high-yield production of properly processed CG54455, stable expression in cells is preferred. Cell lines that stably express CG54455 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG54455 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk-, hgprt- or aprt- cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG54455. Modified culture conditions and media may also be used to enhance production of CG54455. Any techniques known in the art may be applied to establish the optimal conditions for producing CG54455.

An alternative to producing CG54455 or a fragment thereof by recombinant techniques is peptide synthesis. For example, an entire CG54455, or a protein corresponding to a portion of CG54455, can be synthesized by use of a peptide synthesizer. Conventional peptide synthesis or other synthetic protocols well known in the art may be used.

Proteins having the amino acid sequence of CG54455 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is add-labile, and Fmoc, which is base-labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG54455 is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG54455 can be found in Section 6, infra.

### 5.3. Antibodies to CG54455

In various embodiments, monoclonal or polyclonal antibodies specific to CG54455, or a domain of CG54455, can be used in immunoassays to measure the amount of CG54455 or used in immunoaffinity purification of a CG54455 protein. A Hopp & Woods hydrophilic analysis (see Hopp & Woods, Proc. Natl. Acad. Sci. U.S.A. 78:3824-3828 (1981) can be used to identify hydrophilic regions of a protein, and to identify potential epitopes of a CG54455 protein.

The antibodies that immunospecifically bind to an CG54455 or an antigenic fragment thereof can be produced by any method known in the art for the synthesis of antibodies, in particular, by chemical synthesis or preferably, by recombinant expression techniques.

Polyclonal antibodies immunospecific for CG54455 or an antigenic fragment thereof can be produced by various procedures well-known in the art. For example, a CG54455 protein can be administered to various host animals including, but not limited to, rabbits, mice, and rats, to induce the production of sera containing polyclonal antibodies specific for the CG54455. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/01 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non human immuoglobulin. A humanized antibody comprises substantially all of at least one, and typically two, variable domains (Fab, Fab′, F(ab′)2, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489 498; Studnicka et al., 1994, Protein Engineering 7(6):805 814; and Roguska et al., 1994, PNAS 91:969 973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s 5977s (1995), Couto et al., Cancer Res. 55(8):1717 22 (1995), Sandhu J S, Gene 150(2):409 10 (1994), and Pedersen et al., J. Mol. Biol. 235(3):959 73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323.)

Further, the antibodies that immunospecifically bind to CG54455 or an antigenic fragment thereof can, in turn, be utilized to generate anti-idiotype antibodies that “mimic” CG54455 or an antigenic peptide thereof using techniques well-known to those skilled in the art. (See, e.g., Greenspan & Bona, 1989, FASEB J. 7(5):437-444; and Nissinoff, 1991, J. Immunol. 147(8):2429-2438).

### 5.3.1 Polynucleotide Sequences Encoding an Antibody

The invention provides polynucleotides comprising a nucleotide sequence encoding an antibody or fragment thereof that immunospecifically binds to CG54455 or an antigenic fragment thereof. The invention also encompasses polynucleotides that hybridize under high stringency, intermediate, or lower stringency hybridization conditions to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Once the amino acid sequences of a CG54455 or an antigenic fragment thereof is known, nucleotide sequences encoding this antibody or a fragment thereof (e.g., a CDR) can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

### 5.3.2 Recombinant Expression of an Antibody

Recombinant expression of an antibody of the invention, derivative, analog or fragement thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well-known in the art. See, e.g., U.S. Pat. No. 6,331,415. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication No. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715, and those describe in Section 5.2., supra). The expression levels of an antibody molecule can be increased by vector amplification (for a review, see Bebbington and Hentschel. The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.4. Structural Prediction and Functional Analysis of CG54455

Any methods known in the art can be used to determine the identity of a purified CG54455 protein of the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG54455 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

The purity of a CG54455 protein of the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG54455 protein in a composition used in accordance to the instant invention is 50%-100% pure by densitometry, or at least 50%, at least 60%, at least 70%, at least 80%, at least 90%, or at least 95% pure by densitometry.

The biological activities and/or potency of CG54455 of the present invention can be determined by any methods known in the art. For example, compositions for use in therapy in accordance to the methods of the present invention can be tested in suitable cell lines for one or more activities that FGF-22 possesses (e.g., cellular proliferation stimulatory activity). Non-limiting examples of such assays are described in Section 6, infra.

Structure prediction, analysis of crystallographic data, sequence alignment, as well as homology modeling, can also be accomplished using computer software programs available in the art, such as BLAST, CHARMm release 21.2 for the Convex, and QUANTA v.3.3, (Molecular Simulations, Inc., York, United Kingdom). Other methods of structural analysis can also be employed. These include, but are not limited to, X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

The half life of a protein is a measurement of protein stability and indicates the time necessary for a one half reduction in activity of the protein. The half-life of a CG54455 protein can be determined by any method measuring activity of CG54455 in samples from a subject over a period of time. The normalization to concentration of CG54455 in the sample can be done by, e.g., immunoassays using anti-CG54455 antibodies to measure the levels of the CG54455 molecules in samples taken over a period of time after administration of the CG54455, or detection of radiolabelled CG54455 molecules in samples taken from a subject after administration of the radiolabeled CG54455 molecules. In specific embodiments, techniques known in the art can be used to prolong the half life of an CG54455 in vivo. For example, albumin or inert polymer molecules such as high molecular weight polyethyleneglycol (PEG) can be used. See, e.g., International Publication Nos. WO 93/15199, WO 93/15200, and WO 01/77137; and U.S. Pat. No. 6,528,485.

Compositions comprising one more CG54455 for use in a therapy can also be tested in suitable animal model systems prior to testing in humans. To establish an estimate of drug activity in relevant model experiments, an index can be developed that combines observational examination of the animals as well as their survival status. The effectiveness of CG54455 in preventing and/or treating a disease can be monitored by any methods known to one skilled in the art, including but not limited to, clinical evaluation, and measuring the level of CG54455 biomarkers in a biosample.

Any adverse effects during the use of CG54455 alone or in combination with another therapy (e.g., another therapeutic or prophylactic agent) are preferably also monitored. Undesired effects typically experienced by patients taking one or more agents other than CG54455 are numerous and known in the art. Many are described in the Physicians' Desk Reference (58th ed., 2004).

### 5.5. Uses of CG54455

The present invention provides nucleic acids, proteins, and antibodies of CG54455, and their uses in preventing and/or treating a disorder associated with a pathology of epithelial cells. In one embodiment, the present invention provides methods of preventing and/or treating a pathology of epithelial cells comprising administering to a subject in need thereof a composition comprising one or more CG54455 proteins and/or CG54455 nucleic acids. In another embodiment, the present invention provides methods of stimulating proliferation, differentiation or migration of epithelial cells comprising administering to a subject in need thereof an effective amount of a composition comprising one or more CG54455 proteins and/or CG54455 nucleic acids.

Epithelial membranes are continuous sheets of cells with contiguous cell borders that have characteristic specialized sites of close contact called cell junction. Such membrane, which can be one or more cells thick, contain no capillaries. Epithelia are attached to the underlying connective tissue by a component known as a basement membrane, which is a layer of intercellular material of complex composition that is distributed as a thin layer between the epithelium and the connective tissue.

Stratified squamous nonkeratinizing epithelium is common on wet surfaces that are subject to considerable wear and tear at sites where absorptive function is not required. The secretions necessary to keep such surfaces wet have to come from appropriately situated glands. Sites lined by this type of epithelium include the esophagus and the floor and sides of the oral cavity.

Simple columnar epithelium is made up of a single layer of tall cells that again fit together in a hexagonal pattern. In simple secretory columnar epithelium, the columnar cells are all specialized to secret mucus in addition to being protective. Sites of this type of epithelium is present include the lining of the stomach.

A simple columnar epithelium that is made up of absorptive cells as well as secretory cells lines the intestine. To facilitate absorption, this membrane is only one cell thick. Interspersed with cells that are specialized for absorption, there are many goblet cells that secrete protective mucus.

The stromal compartment of the cavities of bone is composed of a net-like structure of interconnected mesenchymal cells. Stromal cells are closely associated with bone cortex, bone trabecule and to the hemopoietic cells. The bone mmarrow-stromal micro- environment, is a complex of cells, extracellular matrix (ECM) with growth factors and cytokines that regulate osteogenesis and hemopoiesis locally throughout the life of the individual. The role of the marrow stroma in creating the microenvironment for bone physiology and hemopoiesis lies in a specific subpopulation of the stroma cells. They differentiate from a common stem cell to the specific lineage each of which has a different role. Their combined function results in orchestration of a 3-D-architecture that maintains the active bone marrow within the bone.

In adults, blood cells are produced by the bone marrow, the spongy material filling the body's bones. The bone marrow produces two blood cell groups, myeloid and lymphoid. The myeloid cell line includes, e.g., the following: (1) Immature cells called erythrocytes that later develop into red blood cells; (2) Blood clotting agents ( platelets); (3) Some white blood cells, including macrophages (which act as scavengers for foreign particles), eosinophils (which trigger allergies and also defend against parasites), and neutrophils (the main defenders against bacterial infections). The lymphoid cell line includes, e.g., the lymphocytes, which are the body's primary infection fighters. Among other vital functions, certain lymphocytes are responsible for producing antibodies, factors that can target and attack specific foreign agents (antigens). Lymphocytes develop in the thymus gland or bone marrow and are therefore categorized as either B-cells (bone marrow-derived cells) or T-cells (thymus gland-derived cells).

According to the present invention, a CG54455 protein and/or nucleic acid can regulate proliferation, differentiation, and/or migration of epithelial cells, and thus have prophylactic and/or therapeutic effects on a disorder associated with a pathology of epithelial cells. A CG54455 protein and/or nucleic acid may also regulate indirectly the proliferation, differentiation, and/or migration of mesenchymal cells via the induction of soluble factors by epithelial cells.

Accordingly, CG54455 may also be used in, e.g., wound and/or bum repairing and healing, ligament repairing, cartilage growth and/or repairing, promoting skin graft growth, increasing bone density, stimulating stem cell growth and/or differentiation, preventing and/or treating stroke, Alzheimer's disease, ischemic heart disease and/or aneurysms, or ulcers.

CG54455 shares high homology to certain known FGFs, which have been shown to play important roles in inflammation, cell proliferative disorders, including but not limited to, cancer, blood vessel formation, and arthritis. In one embodiment, a CG54455 protein and/or nucleic acid is used for detection of inflammatory diseases, including but not limited to, psoriasis, Crohn's disease, and for identification of cell proliferative disorders, including but not limited to, cancer. In another embodiment, a CG54455 protein and/or nucleic acid is a target for therapeutic agents in an inflammatory disease or a cell Proliferative disorder.

Toxicity and therapeutic efficacy of a composition of the invention (e.g., a composition comprising one or more CG54455 proteins) can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio of LD₅₀/ED₅₀. Compositions that exhibit large therapeutic indices are preferred. While compositions that exhibit toxic side effects may be used, care should be taken to design a delivery system that targets such composition to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the ED50 with little or no toxicity. The dosage may vary within this range depending upon the dosage form employed, the route of administration utilized, the severity of the disease, age and weight of the subject, and other factors normally considered by a medical professional (e.g., a physician). For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell cultures. Such information can be used to more accurately determine useful doses in humans. Levels in plasma may be measured, for example, by enzyme linked immunosorbent assays (ELISAs).

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more CG54455 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 25 mg/kg, at least 50 mg/kg, at least 75 mg/kg, or at least 100 mg/kg (as measured by UV assay). In another embodiment, the dosage of a composition comprising one or more CG54455 proteins for administration in a human patient provided by the present invention is between 0.001-100 mg/kg, between 0.001-50 mg/kg, between 0.001-25 mg/kg, between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or by UV absorbance, and the concentration may vary depending on what assay is being used. In a non-limiting example, the protein concentration in a pharmaceutical composition of the instant invention is measured by UV absorbance that uses a direct measurement of the UV absorption at a wavelength of 280 nm, and calibration with a well characterized reference standard of CG54455 protein. Test results obtained with this UV method (using CG54455 reference standard) are three times lower than test results for the same sample(s) tested with the Bradford method.

The appropriate and recommended dosages, formulation and routes of administration for treatment modalities such as chemotherapeutic agents, radiation therapy and biological/immunotherapeutic agents such as cytokines, which can be used in combination with a composition comprising one or more CG54455, are known in the art and described in such literature as the Physician's Desk Reference (58th ed., 2004).

### 5.6. Administration, Pharmaceutical Compositions and Kits

Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG54455 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG54455 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG54455, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g.,arginine in various salt forms, sulfobutyl ether Beta-cyclodextrin sodium, or sucrose), excipient, or vehicle with which CG54455 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG54455 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG54455 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG54455 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG54455 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG54455 may be encapsulated within liposomes or may form complexes thereto, in particular to cationic liposomes. Alternatively, the compositions comprising CG54455 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG54455 can be formulated in an aerosol form, spray, mist or in the form of drops or powder if intranasal administration is preferred. In particular, a composition comprising CG54455 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG54455 proteins may also be formulated into a microcapsule with one or more polymers (e.g., hydroxyethyl starch) form the surface of the microcapsule. Such formulations have benefits such as slow-release.

A composition comprising CG54455 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyomithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG54455 can be delivered to a subject by pulmonary administration, e.g., by use of an inhaler or nebulizer, of a composition formulated with an aerosolizing agent.

In a preferred embodiment, a composition comprising CG54455 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG54455 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG54455 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

The invention also provides kits for carrying out the therapeutic regimens of the invention. Such kits comprise in one or more containers prophylactically or therapeutically effective amounts of the composition of the invention (e.g., a composition comprising one or more CG54455 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG54455 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLES

The present invention is further illustrated by the following non-limiting examples.

### 6.1. Example 1

### Molecular Cloning of the Full Length FGF-22

In this example, cloning is described for the full length FGF-22 clone. Olignucleotide primers were designed to PCR amplify the full length FGF-22 sequence. The forward primers include an in-frame BgIII restriction site: 4301999 TOPO 5′:-AGATCT CCACC ATG CGC CGC CGC CTG TGG CTG GGC CTG-3′ (SEQ ID NO: 31), and 4301999 Forward: 5′-CTCGTC AGATCT CCACC ATG CGC CGC CGC CTG TGG CTG GGC CTG-3′ (SEQ ID NO: 32). The forward primers also include a consensus Kozak sequence (CCACC) upstream to the ATG Start codon.

The reverse primers contain an in-frame XhoI restriction site: 4301999 TOPO: 5′-CTCGAG GGA GAC CAG GAC GGG CAG GAA GTG GGC GGA-3′ (SEQ ID NO: 33) and 4301999 Reverse: 5′-CTCGTC CTCGAG GGA GAC CAG GAC GGG CAG GAA GTG GGC GGA-3′ (SEQ ID NO: 34).

Independent PCR reactions were performed using 5 ng human fetal brain cDNA template and corresponding primer pairs. The reaction mixtures contained 1 μM of each of the 4301999 TOPO Forward and 4301999 TOPO Reverse or 4301999 Forward and 4301999 Reverse primers, 5 micromoles dNTP (Clontech Laboratories, Palo Alto Calif.) and 1 microliter of 50×Advantage-HF 2 polymerase (Clontech Laboratories, Palo Alto Calif.) in 50 microliter volume. The following reaction conditions were used:

| | | |
|---|---|---|
| a) | 96° C. | 3 minutes |
| b) | 96° C. | 30 seconds denaturation |
| c) | 70° C. | 30 seconds, primer annealing. This temperature was |
| | | gradually decreased by 1° C./cycle |
| d) | 72° C. | 1 minute extension. |

Repeat steps b-d 10 times

| | | |
|---|---|---|
| e) | 96° C. | 30 seconds denaturation |
| f) | 60° C. | 30 seconds annealing |
| g) | 72° C. | 1 minute extension |

Repeat steps e-g 25 times

| | | |
|---|---|---|
| h) | 72° C. | 5 minutes final extension |

The expected 510 bp amplified product was detected by agarose gel electrophoresis in both samples. The fragments were purified from agarose gel. The fragment derived from the 4301999 TOPO Forward and 4301999 TOPO Reverse primed reaction was cloned into the pCDNA3.1-TOPO-V5-His vector (Invitrogen, Carlsbad, Calif.). The fragment, derived from the 4301999 Forward and 4301999 Reverse primed reaction was cloned into the pBlgHis vector (CuraGen Corp.). The cloned inserts were sequenced and verified as an open reading frame coding for the predicted full length FGF-22. The cloned sequence was determined to be 100% identical to the predicted sequence.

### 6.2. Example 2

### Molecular Cloning of the Mature Form of FGF-22

In this example, cloning is described for the mature form of the FGF-22 clone (CG54455-06). Using the verified FGF-22 insert from the pCDNA3.1-TOPO-V5-His construct, as template, oliglonucleotide primers were designed to PCR amplify the mature form of FGF-22 PCR reaction was set up to amplify the mature form of FGF-22. The forward primer, FGF-22C forward: 5′-AGATCT ACC CCG AGC GCG TCG CGG GGA CCG-3′ (SEQ ID NO: 35). The reverse primer, 4301999 Reverse: 5′-CTCGTC CTCGAG GGA GAC CAG GAC GGG CAG GAA GTG GGC GGA-3′ (SEQ ID NO: 36).

The PCR reactions were set up using 0.1 ng pCDNA3.1-TOPO-V5-His-FGF-22 plasmid DNA template representing the full length FGF-22, 1 μM of each of the corresponding primer pairs, 5 micromoles dNTP (Clontech Laboratories, Palo Alto Calif.) and 1 microliter of 50×Advantage-HF 2 polymerase (Clontech Laboratories, Palo Alto Calif.) in 50 microliter volume. The following reaction conditions were used:

| a) | 96° C. | 3 minutes denaturation |
|---|---|---|
| b) | 96° C. | 30 seconds denaturation |
| c) | 60° C. | 30 seconds primer annealing |
| d) | 72° C. | 1 minute extension |
| repeat steps b-d 15 times | | |
| e) | 72° C. | 5 minutes final extension |

The expected 450 bp amplified product was detected by agrose gel electrophoresis. The fragments were purified from the agarose gel and ligated to pCR2.1 vector (Invitrogen, Carlsbad, Calif.). The cloned inserts were sequenced and the inserts were verified as open reading frames coding for the predicted mature form of FGF-22 (CG54455-06).

### 6.3. Example 3

### Preparation of the Mammalian Expression Vector pCEP4/Sec

An expression vector, named pCEP4/Sec, was constructed for examining expression of SECX nucleic acid sequences. pCEP4/Sec is an expression vector that allows heterologous protein expression and secretion by fusing any protein to the Ig Kappa chain signal peptide. Detection and purification of the expressed protein are aided by the presence of the V5 epitope tag and 6×His tag at the C-terminus (Invitrogen, Carlsbad, Calif.).

To construct pCEP4/SEC, theoligonucleotide primers, pSec-V5-His Forward: 5′-CTCGTCCTCGAGGGTAAGCCTATCCCTAAC-3′ (SEQ ID NO: 37) and 5′-pSec-V5-His Reverse:CTCGTCGGGCCCCTGATCAGCGGGTTTAAAC-3′ (SEQ ID NO: 38), were designed to amplify a fragment from the pcDNA3.1-V5His (Invitrogen, Carlsbad, Calif.) expression vector that includes V5 and His6. The PCR product was digested with XhoI and ApaI and ligated into the XhoI/ApaI digested pSecTag2 B vector harboring an Ig kappa leader sequence (Invitrogen, Carlsbad Calif.). The correct structure of the resulting vector, pSecV5His, including an in-frame Ig-kappa leader and V5-His6 was verified by DNA sequence analysis. The vector pSecV5His was digested with PmeI and NheI to provide a fragment retaining the above elements in the correct frame. The PmeI-NheI fragment was ligated into the BamHI/Klenow and NheI treated vector pCEP4 (Invitrogen, Carlsbad, Calif.). The resulting vector was named pCEP4/Sec and includes an in-frame Ig kappa leader, a site for insertion of a clone of interest, V5 and His6 under control of the PCMV and/or the PT7 promoter.

### 6.4. Example 4

### Expression of FGF-22 in Human Embryonic Kidney 293 Cells

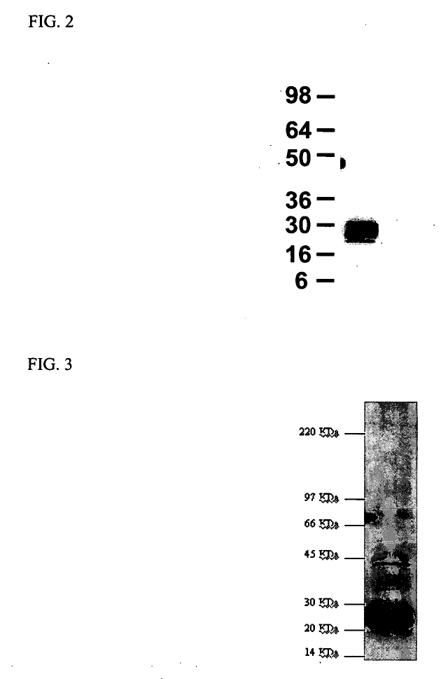

A 0.5 kb BgIII-XhoI fragment containing the FGF-22 sequence (CG54455-03) was isolated from pCR2.1-FGF10-X and subcloned into BamHI-XhoI digested pCEP4/Sec to generate expression vector pCEP4/Sec-FGF10-X. The pCEP4/Sec-FGF10-X vector was transfected into human embryonic kidney 293 cells using the LipofectaminePlus reagent following the manufacturer's instructions (Gibco/BRL). The cell pellet and supernatant were harvested 72 hours after transfection and examined for FGF-22 expression by Western blotting under reducing conditions with an anti-V5 antibody. As shown in FIG. 2, FGF-22 is expressed as an about 27-29 kDa protein secreted by human embryonic kidney 293 cells.

### 6.5. Example 5

### Expression of FGF-22 (Full Length and Mature Form) in Recombinant E. coli

The vector pRSETA (InVitrogen Inc., Carlsbad, Calif.) was digested with XhoI and NcoI restriction enzymes. Oligonucleotide linkers CATGGTCAGCCTAC and TCGAGTAGGCTGAC were annealed at 37° C. and ligated into the XhoI-NcoI treated pRSETA. The resulting vector was confirmed by restriction analysis and sequencing and was named pETMY. The BamHI-XhoI fragment (see above) was ligated into the pETMY that was digested with BamHI and XhoI restriction enzymes. The expression vector was named pETMY-FGF10-X. In this vector, hFGF10-X was fused to the 6×His tag and T7 epitope at its N-terminus. The plasmid pETMY-FGF10-X was then transformed into the E. coli expression host BL21 (DE3, pLys) (Novagen, Madison, Wis.) and the expression induction of protein FGF10-X was carried out according to the manufacturer's instructions. After induction, total cells were harvested, and proteins were analyzed by Western blotting using anti-HisGly antibody (Invitrogen, Carlsbad, Calif.).

Expression and Purification of CG5445S06 in E. coli strain BL21(DE3): A 456 bp long BgIII-XhoI fragment containing the CG54455-06 (mature form of CG54455-01) was subcloned into BamHI-XhoI digested pETMY-His (Invitrogen) to generate plasmid 2021. The resulting plasmid 2021 was transformed into E. coli using the standard transformation protocol. The cells were harvested 2 h post induction with IPTG and disrupted by sonication. The sonicate was brought to a final concentration of 0.5 M NaCl and was passed through a metal chelation column (5 ml Amersham HiTrap metal chelate column). The final protein fraction was eluted using 1× phosphate buffered saline (Mediatech Cellgro, VA) containing 0.4 M NaCl and 500 mM imidazole. Protein samples were stored at 4° C. The expression and purification of CG54455-06 were assessed by Western blot analysis using HRP conjugated anti-His antibody. CG54455-06 was expressed as a 24 kDa protein in E. coli (FIG. 3).

### 6.6. Example 6

### Expression of CG54455-06 in Stable CHO-K1 Cells

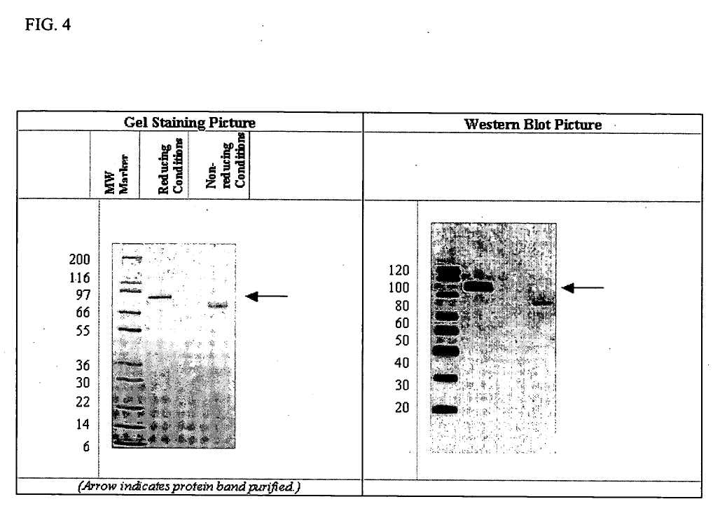

A 456 bp long BgIII-XhoI fragment containing the CG54455-06 (mature form of FGF-22) sequence was subcloned into BamHI-XhoI digested pEE14.4FL2_MSA to generate plasmid 3337. The resulting plasmid 3337 was transfected into CHO-K1 cells using the LipofectaminePlus reagent following the manufacturer's instructions (Invitrogen/Gibco) and stable clones were selected based on resistance against MSX. The culture media was DMEM, 10% FBS, 1× nonessential amino acids. The expression and secretion levels of the clone were assessed by Western blot analysis using HRP conjugated V5 antibody. The V5 epitope is fused to the gene of interest at the Cter, in the pEE14.4Sec vector. FIG. 4 shows that CG54455 is expressed, and a 88 kDa protein is secreted by the CHO-K1 cells.

Purification of CG54455-06 expressed in stable CHO-K1 cells: CG54455-MSA fusion protein enriched conditioned medium (10 L) generated by CHO-K stable transfectants (FIG. 4) was clarified by filtration, and passed through a metal chelation column (50 mL Pharmacia column). The protein was eluted by step elution using buffer containing 25 mM, 50 mM, 100 mM, and 500 mM imidazole. The eluted protein was further subjected to intermediate purification on a 5 ml metal chelation column (Pharmacia) and eluted with a linear gradient from 0 to 500 mM imidazole. The final protein fraction was dialyzed against 20 mM Tris-HCl, pH7.4+150 mM NaCl. Protein samples were stored at −70° C.

### 6.7. Example 7

### Molecular Model of FGF-22

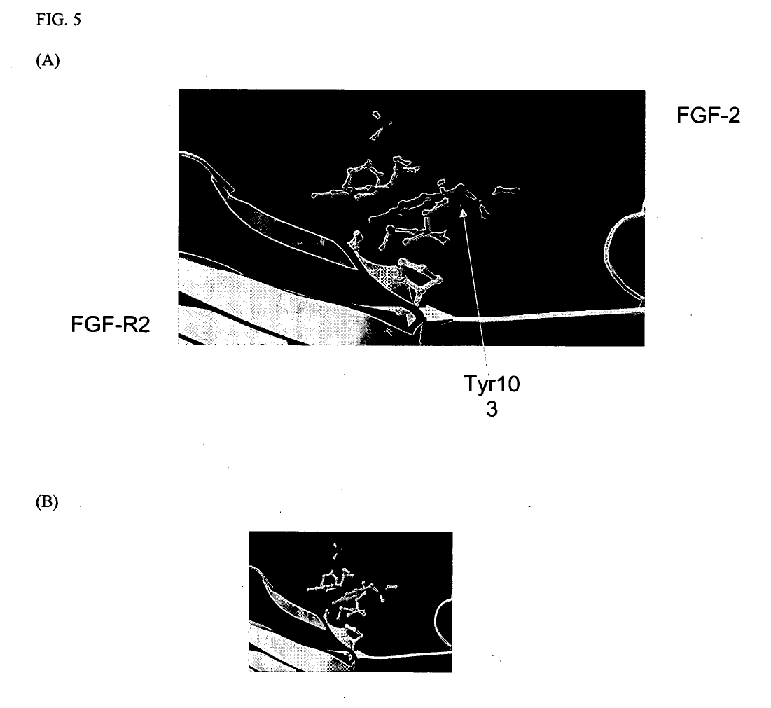

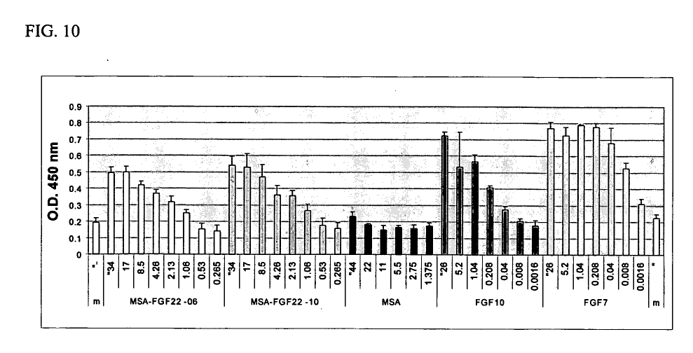

As shown in FIG. 1, other genes that FGF-22 showed high homology to, e.g., hFGF-10, hFGF-7, share a conserved tyrosine (Y) residue at the corresponding position 124 of SEQ ID NO:2, where FGF-22 has a histidine (H) at this position. FGF-22 was modeled in FIG. 5 based on FGF-2 model. Tyr103 of FGF-2 corresponds to His124 of FGF-22. Tyr and His are both planar hydrophobic residues. Non-conserved H124 residue in FGF-22 may cause receptor binding property that is distinct from FGF-7 and FGF-10. Several clones were created for FGF-22 or its mature forms with Tyr substitution at this specific position (e.g. CG54455-10 comprises mature form of FGF-22 with such change). Section 6.8 (see FIG. 10) shows that CG54455 stimulates cell proliferation.

### 6.8. Example 8

### Cellular Proliferation Response to FGF-22

Novel members of the FGF family could have significant therapeutic potential in diseases associated with cell and tissue remodeling, as these growth factors regulate diverse cellular functions such as growth, survival, apoptosis, motility and differentiation (Szebenyi G and Fallon J F, Int Rev Cytol 1999 185:45-106). BrdU incorporation (proliferation assay) was performed to characterize the biological activity of CG54455. Fibroblast growth factors are known to have both stimulatory and inhibitory effects on wide variety of cell types. The proliferative response of BaF3R2b (BaF3 cells expressing the FGFR 2 IIIb receptor) to mature CG54455-06 and CG54455-06 fused to MSA were also evaluated. Baf3 cells that stably express various alpha isoforms of the fibroblast growth factor receptors (FGFRs) (Omitz et al., 1996, Receptor specificity of the Fibroblast growth facotor family. J. Biol. Chem., 271 (25):15292-15297) were also used for evaluation. The cell lines evaluated were BaF3R2b (murine B lymphoma cell line expressing the FGFR 2 IIIb receptor), Balb/MK (murine keratinocyte cell line), CCD1106 (human keratinocyte cell line) and CCD1070sk (dermal fibroblasts).

BrdU Incorporation

Proliferative activity was measured by treatment of cultured BaF3 R2b cells with a conditioned media containing CG54455-fusion protein and measurement of BrDU incorporation during DNA synthesis. CG54455-MSA fusion protein enriched conditioned medium (CM-FGF-22) were generated by incubating the CHO-K transfectants in medium containing 5% FBS for 32 hours (as described above and depicted in FIG. 4). Non-transfected CHO-K cells were used to generate conditioned medium control (CM-CT).

Cells were cultured in RPMI supplemented with 5% fetal bovine serum, beta mercaptoethanol (55 uM), and 5 ug/ml heparin. Purified CG54455-MSA protein or the His-tagged CG54455 and the control (positive control:FGF10 or FGF7 and negative control: MSA) proteins were added at different dilution as shown in the FIG. 4, and the cells were grown for 72 hours. BrdU (10 μM final concentration) was then added and incubated with the cells for 3 h. BrdU incorporation was assayed according to the manufacturer's specifications (Boehringer Mannheim, Indianapolis, Ind.).

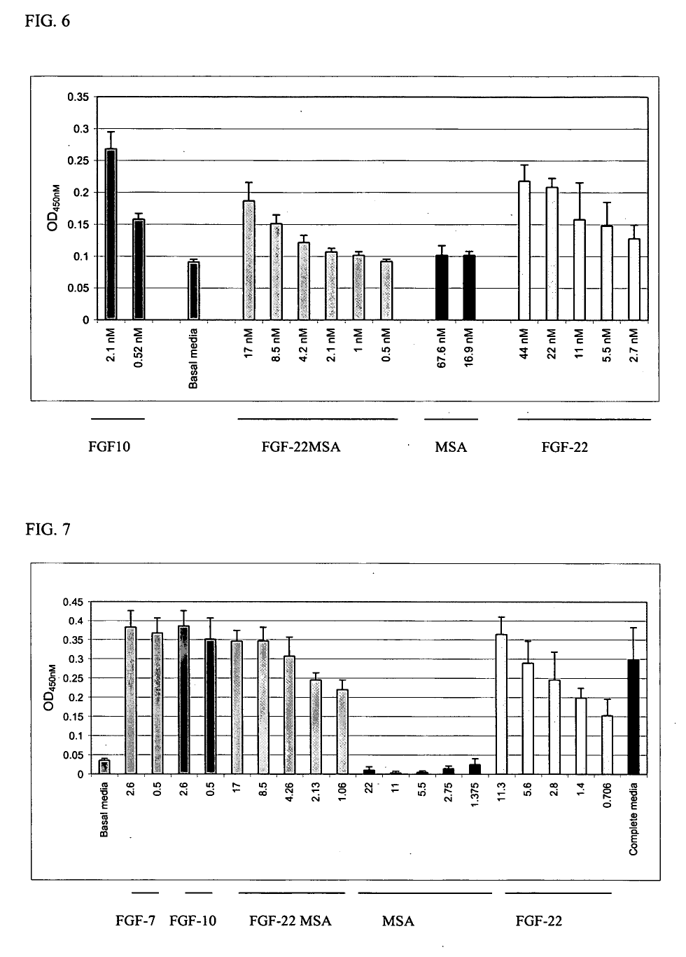

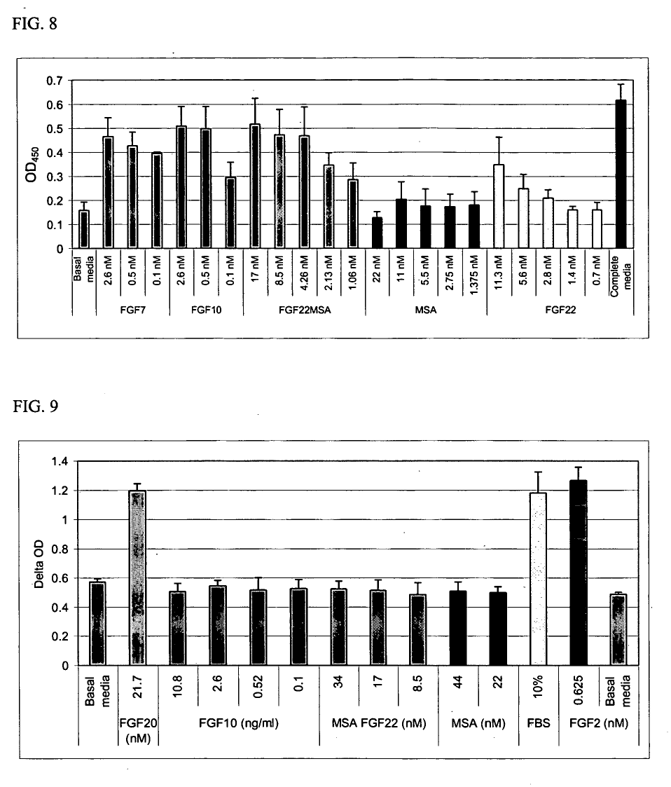

The results from this procedure showed that conditioned medium from MSA-FGF-22 CHO-K1 transfectants and the naked CG54455-06 both demonstrated dose-dependant proliferative activity on BaF3 R2b cells (FIG. 6). The positive control, FGF-10 also showed proliferative activity on BaF3R2b. Similar dose-dependent proliferative response of CG54455 was seen with Balb/MK cells (FIG. 7) and CCD1106 cells (FIG. 8) in the presence of comparable concentrations of CG54455-06. However, presence of CG54455-06-MSA did not affect the proliferation of dermal fibroblast cells (CCD1070sk cells) (FIG. 9). CG54455-06 (wildtype) and CG54455-10 (¹²⁴H→Y mutant) proteins showed similar proliferative effect on Balb/MK cells.

### 6.9. Example 9

### Interaction of FGF-22 with FGF Receptors

A proliferation assay was used to determine the receptor specificity of CG54455. Recombinant Baf3 cells (transfectant cells generated by Omitz et al)expressing either murine FGF receptors FGFR1 IIIc, FGFR2 IIIc, FGFR3 IIIb, FGFR3 IIIc or human FGFR2 IIIb were assayed for proliferation in the presence of FGF10, CG54455-06, CG54455-06-MSA.

The results shown in FIG. 11 indicate that FGF22 signals through FGFR2 IIIb receptor similar to that of FGF10.

Receptor neutralization on proliferative effect of CG54455-06, FGF-10 and FGF-1 on Baf3R2IIIb cells were carried out using soluble FGF receptors 1, 2, 3 or 4. Alamar blue (Biosource International, Calif.) or Cell titer blue (Promega Corporation) was added to the cell culture according to the manufacturer's specifications. Change in color by the indicator dye was measured at 570/630 nm.

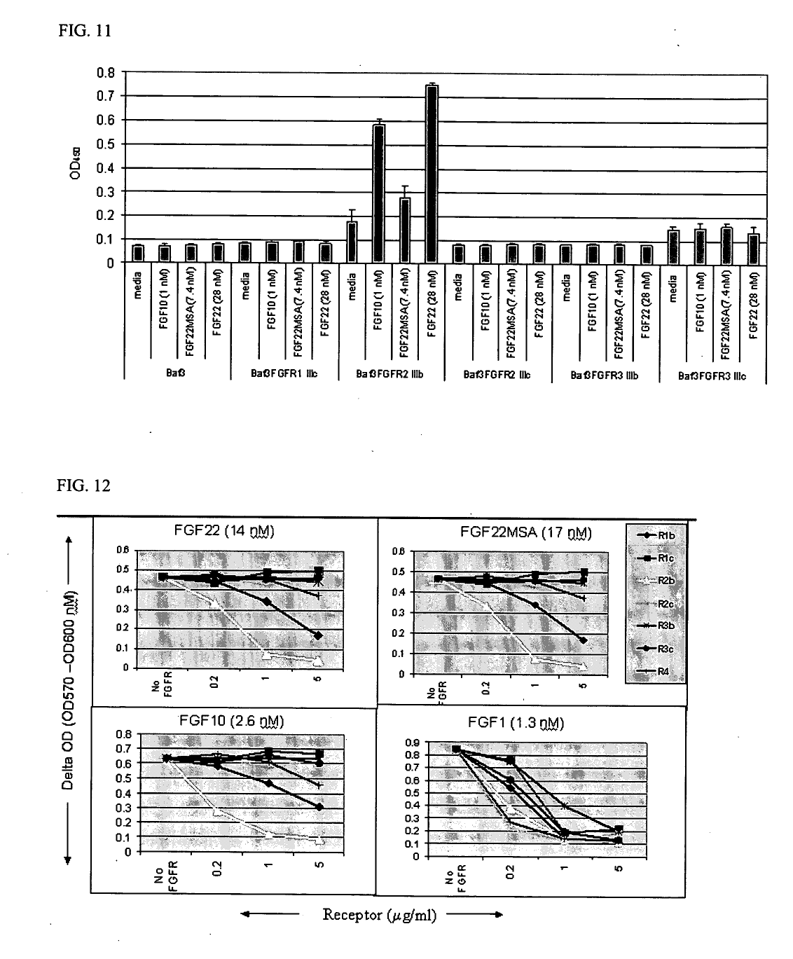

The results indicate that FGF22 signals through the FGFR2 IIIb and FGFR1 IIIb (FIG. 12).

From the results presented, it is postulated that FGF may play an important role in epithelial tissue repair (wound healing) and cytoprotection. The mechanism of action is also suggestive of therapeutic use of FGF22 in other pathologies related to tissue damage such as those associated with ulcers, mucositis, cancer, radiation exposure, arthritis and neuronal damage.

### 6.10. Example 10

### Expression of CG54455-14

A 0.450 kb NdeI-XhoI fragment containing the CG54455-14 sequence was subcloned into NdeI-XhoI digested pETMY (Invitrogen) to generate plasmid 4043. The resulting plasmid 4043 was transformed into E. coli BL21 (DE3) using the standard transformation protocol. The cell pellet and supernatant were harvested 2 hours post induction with IPTG and examined for CG54455-14 expression by Western blot (reducing conditions) using an anti-FGF22 (Anti-FGF22 B4980 polyclonal antibodies; 1:10,000 dilution) or anti-FGF10 (Abcam) antibodies. CG54455-14 is expressed as a 17 kDa protein.

### 6.11. Example 11

### Purification of CG54455-14

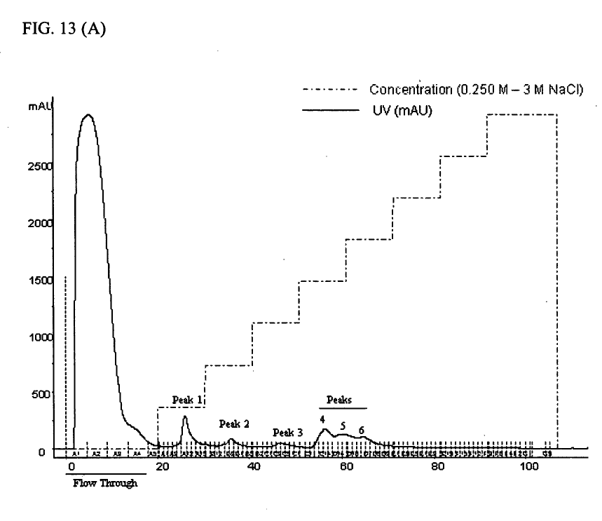

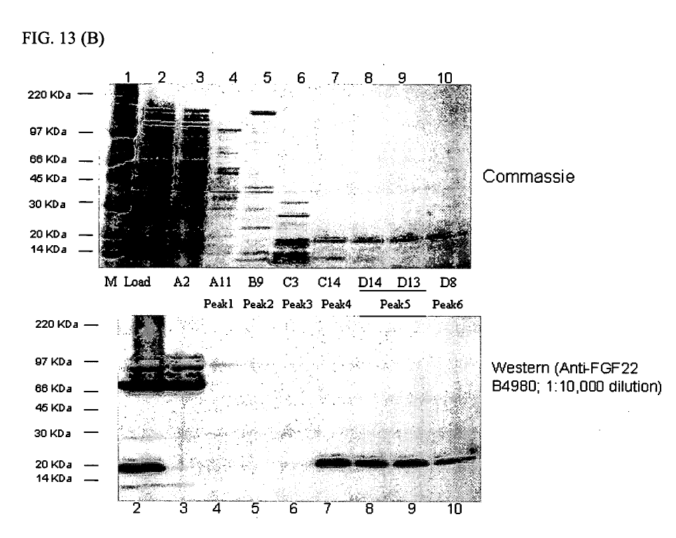

Plasmid 4043 transformed E. coli expressing CG54455-14 protein was grown up in LB or super broth (60L fermentation by New Brunswick Sci. co) and the expression of CG54455-14 was induced with IPTG. The cells were collected by centrifugation, aliquoted as 20g/ 50 ml tube and stored at −80° C. The pellets were resuspended in a 5% W/V ratio in lysis buffer (10 mM sodium phosphate, pH 7.4, 25 mM NaCl) and lysed using microfluidizer (4 cycle at 12,000 PSI). Cell debris was removed by centrifugation at 12,000 RPM in SLA1500 rotor for 10 min×3 cycle, and lysate either stored at −80° C. or used for purification. Lysate was diluted 5 times with binding buffer (25 mM sodium phosphate, pH 7.4, 250 mM sodium chloride), filtered through 0.2 μm filter and applied directly to a heparin-Sepharose affinity column (HSAC) (Hi-Trap, 5 ml bed volume) (Amersham). The column was washed with 4 column volume (20 ml) of binding buffer and subjected to step elution with increment of 12.5% of elution buffer (25 mM sodium phosphate, pH 7.4, 3 M sodium chloride) up to 8 times. The elution profile is shown in FIG. 13A.

As determined by Western blot analysis (FIG. 13B), recombinant FGF-22 was eluted between 1.375-1.8 M NaCl and was about 80% pure and the peak fraction had mitogenic activity in Baf3R2b transfectant and keratinocytes (see section on biological activities of CG54455-14). Subsequent purification of the HSAC material with different HIC columns, cation exchange column for e.g., SP sepharose, and gel filtration column has been studied. CG54455-14 binds tightly to phenyl sepharose, SP sepharose, Superdex 75 and octyl FF column. Preliminary results using 1 ml Butyl FF column (HiTrap, Amersham) showed that FGF22 could bind to butyl FF column and could be eluted with TrisHCl, pH 9.0.

### 6.12. Example 12

### CG54455-14 Induces Proliferation of BaF3R2b Transfectants

CG54455-14 was tested for its activity on BaF3 transfectant cells generated by Ornitz et al which express either the murine FGFR1IIIb, FGFR1IIIc, FGFR3IIIb, FGFR3IIIc FGF2IIIc or human FGFR2IIIb. Cell proliferation was measured by the CellTiter-Blue TM Cell viability assay.

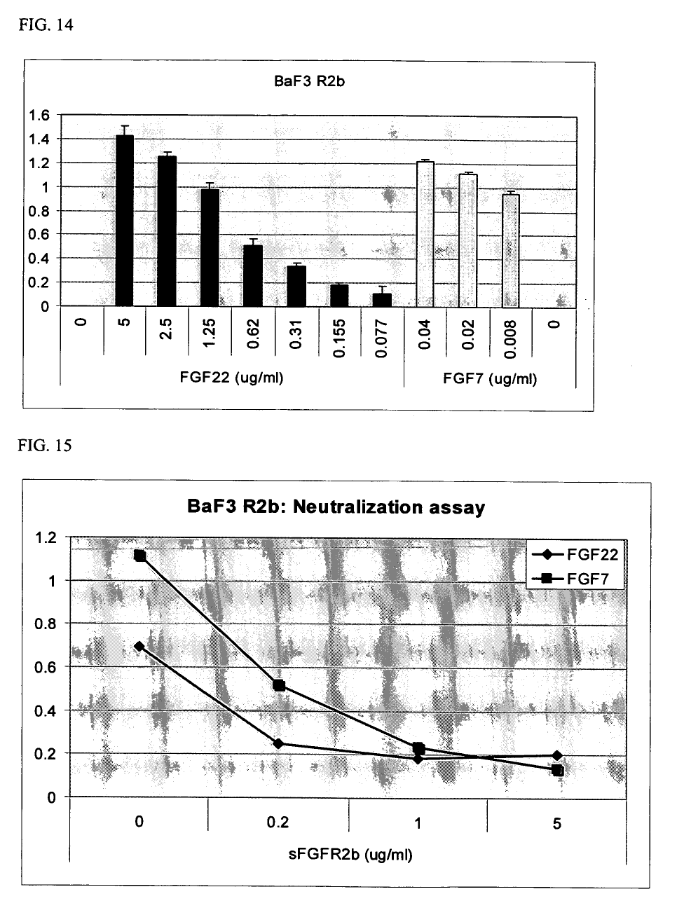

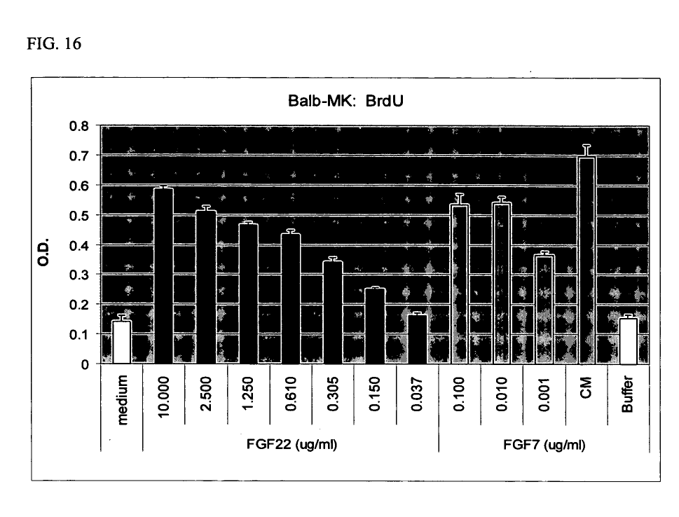

CG54455-14 induced the proliferation of BAF3R2b in a dose dependent fashion (FIG. 14). The proliferative activity of CG54455-14 on BaF3R2b was abrogated by the addition of soluble recombinant FGFR2 IIIb to the culture (R&D systems) (FIG. 15). This indicates that CG54455-14 protein activity is mediated directly through FGFR2b. FGF7 (=KGF1) was used as a positive control.

### 6.13. Example 13

### CG54455-14 Induces Proliferation of Murine Keratinocytes, Balb-MK

CG54455-14 was tested on the murine keratinocyte cell line, Balb-MK. Cell proliferation was determined by BrdU incorporation using an ELISA assay (Roche diagnostics).

CG54455-14 induced the proliferation of Balb/Mk cells in a dose dependent fashion (FIG. 16). FGF7 was used as a positive control in this assay. CG54455-06 and CG54455-10 also induce proliferation of Balb/MK cells in a dos dependent fashion (FIG. 10).

### 6.14. Example 14

### CG4455-14 Activates p42144 MAPK and p70S6 Kinase in Balb-MK Cells

We have shown that CG54455 (-06 and now -14) stimulates the proliferation of keratinocytes. However, little is known about the signaling pathways involved. We investigated the role of ERK (p42/44 MAP Kinase) and PI3 Kinase pathways on CG54455 induced proliferation.

CG54455-14 was tested directly for its activity on ERK activation. Briefly, CG54455-14 was added to Balb/Mk for various periods of time (from 5 minutes to 90 minutes). Cells were then lysed in cold buffer containing proteases inhibitors and phosphatase inhibitors. Same amount of cell extracts was then submitted to western blot analysis and activation of ERK and p70S6 kinase was evaluated by phosphorylation of these kinases using anti-p42/44 MAPK and anti-P p70S6 kinase from cell Signaling Technology.

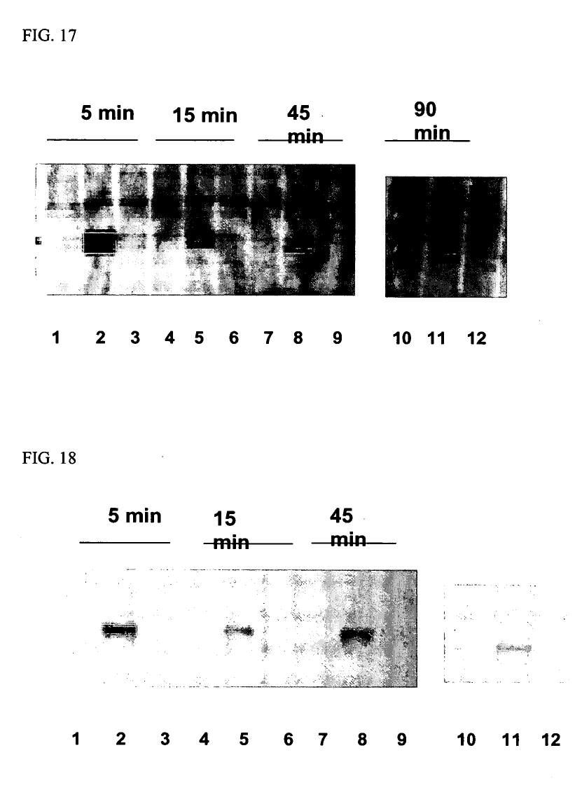

Data showed that CG54455-14 stimulates p42/44 MAPK activation as demonstrated by protein phosphorylation (FIG. 17). In addition, the data also showed that CG54455-14 stimulated p70S6 kinase (FIG. 18), a kinase that is required for cell growth and G1 cell cycle progression

To further determine the role of ERK and PI3 Kinase in the proliferative activity of CG54455-14, selective inhibitors of MAPK and PI3 kinase were tested on Balb/Mk proliferation. The following inhibitors were tested: PD89059, an inhibitor of MEK1/2 activation, U 0126, an inhibitor of MEK1/2 activity, and LY294002, an inhibitor of Pi3 kinase. SB 203580, a selective inhibitor of p38 MAPK, was also tested. Proliferation of Balb/MK cells upon CG54455-14 treatment was determined by CellTiter Blue assay.

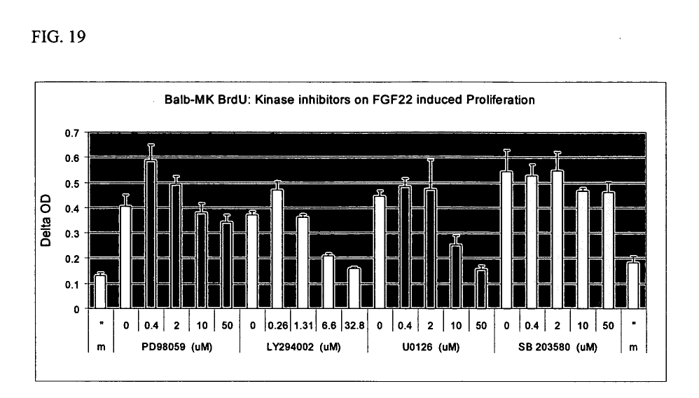

The proliferative activity of CG54455-14 on Balb-Mk cells was abrogated by the addition of MEK1/2 selective inhibitors but not by p38 MAPK inhibitors (FIG. 19). In addition pretreatment of Balb-MK cells by PI3 kinase inhibitor dramatically reduced the effect of CG54455-14.

Together, these data indicate that CG54455-14 binds to and activates FGFR2b (also called KGF receptor). The data also show that CG54455-14 induces the proliferation of keratinocytes and utilizes ERK and possibly PI3kinase pathways to exert its activity.

### 6.15. Example 15

### Quantitative Expression Analysis of CG54455

The quantitative expression of CG54455 was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturers instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C. greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_Screening_Panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1D included 2 control wells and 94 test samples of RNA (Panel 4R) or cDNA (Panels 4D and 4.1 D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma ˜20-50 ng/ml, IL-4 ˜5-10 ng/ml, IL-9 ˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media (DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10−5 M (Gibco), and 10 mM Hepes (Gibco)) and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of −2×106 cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with −50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+and CD19+ cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4 +and CD8+ lymphocytes were cultured in culture media at 106 cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 44 days before RNA was prepared.

B cells were prepared from minced and sieved tonsil tissue (NDRI). Tonsil cells were pelleted and resupended at 106 cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 105-106 cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 47 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×105 cells/ml with 0.1 mM dbcAMP for 8 days, changing the media every 3 days and adjusting the cell concentration to 5×105 cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 107 cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI_Comprehensive Panel_v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N.J.), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta ˜1-1-0 ng/ml, TNF alpha ˜5-50 ng/ml, or prostaglandin E2 for Nhost cells) for 1, 6, 18 or 24 h. All these cells were starved for at least 5 h and cultured in their corresponding basal medium with ˜0.1 to 1% FBS.

Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDRI (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1beta (10 ng/ml), IL-1 beta+TNF-alpha (50 ng/ml), IL-1 beta+Oncostatin (50 ng/ml) and PMA (100 ng/ml). Supernatants were collected and analyzed for MMP 1, 3 and 13 production. RNA was prepared from these samples using standard procedures.

Panels 5D and 5I

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18 - 40 years), otherwise healthy women with and without gestational diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (Pl), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegenerabon V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimers like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

Expression of CG54455 was assessed using the primer-probe sets Ag4346, Ag4347 and Ag7772, described in Tables 4, 5 and 6. Results of the RTQ-PCR runs are shown in Tables 7-14.

TABLE 4
Probe Name Ag4346

| | | | Start | |
|---|---|---|---|---|
| Primers | Sequences | Length | Postion | SEQ ID No |
| Forward | 5′-cgtggtcatcaaagcagtgt-3′ | 20 | 249 | 39 |
| Probe | TET-5′-ctcaggcttctacgtggccatgaac-3′-TAMRA | 25 | 270 | 40 |
| Reverse | 5′-tgcagtccacggtgtagagt-3′ | 20 | 321 | 41 |

TABLE 5
Probe Name Ag4347

| | | | Start | |
|---|---|---|---|---|
| Primers | Sequences | Length | Position | SEQ ID No |
| Forward | 5′-tggagatccgctctgtacacg-3′ | 21 | 221 | 42 |
| Probe | TET-5′-cctgaggacactgctttgatgaccacgt-3′-TAMRA | 26 | 249 | 43 |
| Reverse | 5′-cggttcatggccacgtaga-3′ | 19 | 278 | 44 |

TABLE 6
Probe Name Ag7772

| | | | Start | |
|---|---|---|---|---|
| Primers | Sequences | Length | Postion | SEQ ID No |
| Forward | 5′-tggagatccgctctgtacac-3′ | 21 | 221 | 42 |
| Probe | TET-5′-tcatcaaagcagtgtcctcaggcttc-3′-TAMRA | 26 | 254 | 46 |
| Reverse | 5′-tgcagtccacggtgtagagt-3′ | 20 | 321 | 47 |

TABLE 7
AI_comprehensive panel_v1.0
Column A - Rel. Exp.(%) Ag4347, Run 278182082

| Tissue Name | A | Tissue Name | A |
|---|---|---|---|
| 110967 COPD-F | 10.0 | 112427 Match Control Psoriasis-F | 60.3 |
| 110980 COPD-F | 19.2 | 112418 Psoriasis-M | 12.1 |
| 110968 COPD-M | 13.3 | 112723 Match Control Psoriasis-M | 0.8 |
| 110977 COPD-M | 59.5 | 112419 Psoriasis-M | 4.9 |
| 110989 Emphysema-F | 16.4 | 112424 Match Control Psoriasis-M | 15.7 |
| 110992 Emphysema-F | 14.3 | 112420 Psoriasis-M | 15.2 |
| 110993 Emphysema-F | 6.2 | 112425 Match Control Psoriasis-M | 30.1 |
| 110994 Emphysema-F | 4.1 | 104689 (MF) OA Bone-Backus | 21.5 |
| 110995 Emphysema-F | 19.3 | 104690 (MF) Adj “Normal” Bone-Backus | 10.9 |
| 110996 Emphysema-F | 9.3 | 104691 (MF) OA Synovium-Backus | 11.4 |
| 110997 Asthma-M | 1.7 | 104692 (BA) OA Cartilage-Backus | 27.4 |
| 111001 Asthma-F | 4.5 | 104694 (BA) OA Bone-Backus | 5.4 |
| 111002 Asthma-F | 15.7 | 104695 (BA) Adj “Normal” Bone-Backus | 36.1 |
| 111003 Atopic Asthma-F | 14.0 | 104696 (BA) OA Synovium-Backus | 2.0 |
| 111004 Atopic Asthma-F | 21.5 | 104700 (SS) OA Bone-Backus | 4.7 |
| 111005 Atopic Asthma-F | 6.4 | 104701 (SS) Adj “Normal” Bone-Backus | 15.0 |
| 111006 Atopic Asthma-F | 5.1 | 104702 (SS) OA Synovium-Backus | 10.6 |
| 111417 Allergy-M | 18.7 | 117093 OA Cartilage Rep7 | 7.2 |
| 112347 Allergy-M | 4.8 | 112672 OA Bone5 | 2.5 |
| 112349 Normal Lung-F | 2.7 | 112673 OA Synovium5 | 3.4 |
| 112357 Normal Lung-F | 100.0 | 112674 OA Synovial Fluid cells5 | 9.9 |
| 112354 Normal Lung-M | 8.3 | 117100 OA Cartilage Rep14 | 2.7 |
| 112374 Crohns-F | 19.9 | 112756 OA Bone9 | 18.7 |
| 112389 Match Control Crohns-F | 33.9 | 112757 OA Synovium9 | 4.3 |
| 112375 Crohns-F | 6.7 | 112758 OA Synovial Fluid Cells9 | 4.7 |
| 112732 Match Control Crohns-F | 31.4 | 117125 RA Cartilage Rep2 | 3.1 |
| 112725 Crohns-M | 9.1 | 113492 Bone2 RA | 5.8 |
| 112387 Match Control Crohns-M | 2.2 | 113493 Synovium2 RA | 1.8 |
| 112378 Crohns-M | 4.3 | 113494 Syn Fluid Cells RA | 8.0 |
| 112390 Match Control Crohns-M | 28.7 | 113499 Cartilage4 RA | 2.6 |
| 112726 Crohns-M | 1.4 | 113500 Bone4 RA | 10.4 |
| 112731 Match Control Crohns-M | 15.3 | 113501 Synovium4 RA | 5.6 |
| 112380 Ulcer Col-F | 4.4 | 113502 Syn Fluid Cells4 RA | 5.3 |
| 112734 Match Control Ulcer Col-F | 18.8 | 113495 Cartilage3 RA | 5.9 |
| 112384 Ulcer Col-F | 7.0 | 113496 Bone3 RA | 3.5 |
| 112737 Match Control Ulcer Col-F | 1.2 | 113497 Synovium3 RA | 0.4 |
| 112386 Ulcer Col-F | 19.6 | 113498 Syn Fluid Cells3 RA | 1.6 |
| 112738 Match Control Ulcer Col-F | 1.0 | 117106 Normal Cartilage Rep20 | 4.2 |
| 112381 Ulcer Col-M | 0.0 | 113663 Bone3 Normal | 0.0 |
| 112735 Match Control Ulcer Col-M | 0.0 | 113664 Synovium3 Normal | 0.0 |
| 112382 Ulcer Col-M | 7.1 | 113665 Syn Fluid Cells3 Normal | 3.6 |
| 112394 Match Control Ulcer Col-M | 11.9 | 117107 Normal Cartilage Rep22 | 3.3 |
| 112383 Ulcer Col-M | 8.4 | 113667 Bone4 Normal | 10.7 |
| 112736 Match Control Ulcer Col-M | 29.1 | 113668 Synovium4 Normal | 16.4 |
| 112423 Psoriasis-F | 1.0 | 113669 Syn Fluid Cells4 Normal | 4.2 |

TABLE 8
Cellular OA/RA
Column A - Rel. Exp.(%) Ag4346, Run 406013342

| Tissue Name | A | Tissue Name | A |
|---|---|---|---|
| 158667 Nhost medium 1 h | 8.5 | 164336 SW1353 + TNF-a (100 ng/ml) 6 h | 24.5 |
| 158670 Nhost + IL-1b (10 ng/ml), | 3.5 | 164337 SW1353 medium alone 18 h | 49.7 |
| 1 h | | | |
| 158673 Nhost + PGE2 (10-6 M) | 18.2 | 164338 SW1353 + IL-1b (1 ng/ml) 18 h | 38.2 |
| 1 h | | | |
| 158668 Nhost medium alone 6 h | 26.6 | 164339 SW1353 + IL-1b (10 ng/ml) 18 h | 34.9 |
| 158671 Nhost + IL-1b (10 ng/ml) | 29.3 | 164340 SW1353 + TNF-a (10 ng/ml) 18 h | 19.9 |
| 6 h | | | |
| 158674 Nhost + | 2.3 | 164341 SW1353 + IL-1b (100 ng/ml) 18 h | 55.1 |
| PGE2 (10-6 M) 6 h | | | |
| 158669 Nhost medium alone 24 h | 0.0 | 173326 HFLS-RA (cell aplication) medium | 12.2 |
| | | alone 18 h | |
| 158672 Nhost + IL-1b (10 ng/ml) | 33.7 | 173327 HFLS-RA (cell aplication) + TNF-a 18 h | 36.9 |
| 24 h | | | |
| 158675 Nhost + PGE2 (10-6 M) | 41.5 | 173331 MH7A (synoviocyte cell line) medium | 36.6 |
| 24 h | | 1 h | |
| 164327 SW1353 medium alone | 66.0 | 173332 MH7A (synoviocyte cell line) + IL1b | 15.3 |
| 1 h | | 1 h | |
| 164328 SW1353 + IL-1b (1 ng/ml) | 22.5 | 173334 MH7A (synoviocyte cell line) TNFa 1 h | 0.0 |
| 1 h | | | |
| 164329 SW1353 + | 26.8 | 173336 MH7A (synoviocyte cell line) medium | 100.0 |
| IL-1b (10 ng/ml) 1 h | | alone 6 h | |
| 164330 SW1353 + | 20.4 | 173339 MH7A (synoviocyte cell line) + IL1b | 77.9 |
| TNF-a (10 ng/ml) 1 h | | 6 h | |
| 164331 SW1353 + | 51.1 | 173341 MH7A (synoviocyte cell line) TNFa 6 h | 7.6 |
| TNF-a (100 ng/ml) 1 h | | | |
| 164332 SW1353 medium alone | 42.6 | 173342 MH7A (synoviocyte cell line) medium | 15.1 |
| 6 h | | alone 18 h | |
| 164333 SW1353 + IL-1b (1 ng/ml) | 41.5 | 173344 MH7A (synoviocyte cell line) + IL1b | 30.8 |
| 6 h | | 18 h | |
| 164334 SW1353 + | 88.3 | 173346 MH7A (synoviocyte cell line) TNF-a | 25.2 |
| IL-1b (10 ng/ml) 6 h | | 18 h | |
| 164335 SW1353 + | 34.4 | | |
| TNF-a (10 ng/ml) 6 h | | | |

TABLE 9
General_screening_panel_v1.4
Column A - Rel. Exp.(%) Ag4347, Run 222523511

| Tissue Name | A | Tissue Name | A |
|---|---|---|---|
| Adipose | 0.0 | Renal ca. TK-10 | 15.2 |
| Melanoma* | 0.0 | Bladder | 4.3 |
| Hs688(A).T | | | |
| Melanoma* | 3.2 | Gastric ca. (liver met.) NCI-N87 | 8.7 |
| Hs688(B).T | | | |
| Melanoma* M14 | 5.6 | Gastric ca. KATO III | 3.7 |
| Melanoma* LOXIMVI | 3.7 | Colon ca. SW-948 | 1.7 |
| Melanoma* | 2.3 | Colon ca. SW480 | 11.1 |
| SK-MEL-5 | | | |
| Squamous cell | 0.0 | Colon ca.* (SW480 met) SW620 | 12.6 |
| carcinoma SCC-4 | | | |
| Testis Pool | 20.4 | Colon ca. HT29 | 4.7 |
| Prostate ca.* | 5.9 | Colon ca. HCT-116 | 40.1 |
| (bone met) PC-3 | | | |
| Prostate Pool | 2.1 | Colon ca. CaCo-2 | 3.6 |
| Placenta | 18.9 | Colon cancer tissue | 0.0 |
| Uterus Pool | 2.5 | Colon ca. SW1116 | 29.7 |
| Ovarian ca. OVCAR-3 | 8.6 | Colon ca. Colo-205 | 0.0 |
| Ovarian ca. SK-OV-3 | 100.0 | Colon ca. SW-48 | 0.0 |
| Ovarian ca. OVCAR-4 | 7.4 | Colon Pool | 3.9 |
| Ovarian ca. OVCAR-5 | 41.8 | Small Intestine Pool | 10.5 |
| Ovarian ca. IGROV-1 | 13.3 | Stomach Pool | 3.7 |
| Ovarian ca. OVCAR-8 | 27.4 | Bone Marrow Pool | 18.2 |
| Ovary | 27.4 | Fetal Heart | 0.0 |
| Breast ca. MCF-7 | 0.0 | Heart Pool | 1.1 |
| Breast ca. | 17.1 | Lymph Node Pool | 17.4 |
| MDA-MB-231 | | | |
| Breast ca. BT 549 | 10.0 | Fetal Skeletal Muscle | 0.0 |
| Breast ca. T47D | 83.5 | Skeletal Muscle Pool | 9.3 |
| Breast ca. MDA-N | 10.2 | Spleen Pool | 5.1 |
| Breast Pool | 4.5 | Thymus Pool | 9.9 |
| Trachea | 14.4 | CNS cancer (glio/astro) U87-MG | 14.6 |
| Lung | 0.0 | CNS cancer (glio/astro) U-118-MG | 32.3 |
| Fetal Lung | 0.0 | CNS cancer (neuro;met) SK-N-AS | 15.7 |
| Lung ca. NCI-N417 | 2.5 | CNS cancer (astro) SF-539 | 3.6 |
| Lung ca. LX-1 | 15.7 | CNS cancer (astro) SNB-75 | 8.7 |
| Lung ca. NCI-H146 | 0.0 | CNS cancer (glio) SNB-19 | 3.9 |
| Lung ca. SHP-77 | 14.0 | CNS cancer (glio) SF-295 | 54.3 |
| Lung ca. A549 | 6.8 | Brain (Amygdala) Pool | 70.7 |
| Lung ca. NCI-H526 | 4.4 | Brain (cerebellum) | 26.1 |
| Lung ca. NCI-H23 | 17.1 | Brain (fetal) | 40.3 |
| Lung ca. NCI-H460 | 0.0 | Brain (Hippocampus) Pool | 34.4 |
| Lung ca. HOP-62 | 8.8 | Cerebral Cortex Pool | 52.5 |
| Lung ca. NCI-H522 | 12.9 | Brain (Substantia nigra) Pool | 53.6 |
| Liver | 1.2 | Brain (Thalamus) Pool | 57.8 |
| Fetal Liver | 0.0 | Brain (whole) | 31.9 |
| Liver ca. HepG2 | 5.1 | Spinal Cord Pool | 34.9 |
| Kidney Pool | 13.6 | Adrenal Gland | 1.3 |
| Fetal Kidney | 8.6 | Pituitary gland Pool | 2.1 |
| Renal ca. 786-0 | 11.5 | Salivary Gland | 7.4 |
| Renal ca. A498 | 3.5 | Thyroid (female) | 0.0 |
| Renal ca. ACHN | 8.2 | Pancreatic ca. CAPAN2 | 1.8 |
| Renal ca. UO-31 | 0.0 | Pancreas Pool | 0.0 |

TABLE 10
Mini tissue OA/RA
Column A - Rel. Exp.(%) Ag4346, Run 406107070

| Tissue Name | A | Tissue Name | A |
|---|---|---|---|
| 161315 OA PT 5 Cartilage | 2.4 | 161314 OA PT 5 Bone | 2.4 |
| 161291 OA PT 7 Cartilage | 0.4 | 161292 OA PT 7 Bone | 2.0 |
| 161303 OA PT 8 Cartilage | 0.8 | 161302 OA PT 8 Bone | 1.1 |
| 161287 OA PT 10 Cartilage | 0.4 | 161288 OA PT 10 Bone | 3.6 |
| 173546 RA Cartilage PT 85 | 3.4 | 173548 RA PT 85 Bone | 0.6 |
| 161316 OA PT 5 Synovium | 0.4 | 150168 Adrenal gland | 4.2 |
| 161290 OA PT 7 Synovium | 1.3 | 150171 Brain (whole) | 100.0 |
| 161304 OA PT 8 Synovium | 0.5 | 154975 Colon | 4.4 |
| 161289 OA PT 10 Synovium | 2.7 | 154947 Heart | 0.0 |
| 161237 RA PT 1 Synovium | 1.8 | 155689 Kidney pool | 8.4 |
| 173547 RA PT 85 Synovium | 5.0 | 150176 Lung* | 2.9 |
| 173553 RA Synovium Ardais RNA 1 | 6.8 | 150178 Ovary* | 7.9 |
| 173554 RA Synovium Ardais RNA 2 | 1.8 | 144488 Skeletal muscle pool* | 0.0 |
| 173543 Normal Synovium 1 NDRI | 5.6 | 154967 Small intestine | 8.0 |
| 173545 Normal Synovium 3 NDRI | 2.1 | 154970 Stomach* | 0.6 |
| 173555 Normal Synovium Ardais RNA 1 | 20.6 | | |

TABLE 11
PGI1.0
Column A - Rel. Exp.(%) Ag4346, Run 416198456

| Tissue Name | A | Tissue Name | A |
|---|---|---|---|
| 162191 Normal Lung 1 (IBS) | 0.5 | 162185 Emphysema Lung 12 (Ardais) | 0.0 |
| 162570 Normal Lung 4 (Aastrand) | 0.0 | 162184 Emphysema Lung 13 (Ardais) | 1.1 |
| 160468 MD lung | 0.0 | 162183 Emphysema Lung 14 (Ardais) | 6.6 |
| 156629 MD Lung 13 | 0.0 | 162188 Emphysema Lung 15 (Genomic | 82.4 |
| | | Collaborative) | |
| 162571 Normal Lung 3 (Aastrand) | 0.4 | 162177 NAT UC Colon 1(Ardais) | 0.4 |
| 162186 Fibrosis Lung 1 (Genomic | 100.0 | 162176 UC Colon 1(Ardais) | 0.0 |
| Collaborative) | | | |
| 162187 Fibrosis Lung 2 (Genomic | 47.6 | 162179 NAT UC Colon 2(Ardais) | 0.0 |
| Collaborative) | | | |
| 151281 Fibrosis lung 11(Ardais) | 3.5 | 162178 UC Colon 2(Ardais) | 0.0 |
| 162190 Asthma Lung 4 (Genomic | 33.2 | 162181 NAT UC Colon 3(Ardais) | 0.9 |
| Collaborative) | | | |
| 160467 Asthma Lung 13 (MD) | 0.0 | 162180 UC Colon 3(Ardais) | 0.2 |
| 137027 Emphysema Lung 1 (Ardais) | 0.0 | 162182 NAT UC Colon 4 (Ardais) | 3.1 |
| 137028 Emphysema Lung 2 (Ardais) | 3.7 | 137042 UC Colon 1108 | 0.0 |
| 137040 Emphysema Lung 3 (Ardais) | 2.8 | 137029 UC Colon 8215 | 0.6 |
| 137041 Emphysema Lung 4 (Ardais) | 0.0 | 137031 UC Colon 8217 | 0.6 |
| 137043 Emphysema Lung 5 (Ardais) | 0.5 | 137036 UC Colon 1137 | 1.1 |
| 142817 Emphysema Lung 6 (Ardais) | 1.9 | 137038 UC Colon 1491 | 0.8 |
| 142818 Emphysema Lung 7 (Ardais) | 2.6 | 137039 UC Colon 1546 | 0.7 |
| 142819 Emphysema Lung 8 (Ardais) | 5.9 | 162594 NAT Crohn's 47751 (NDRI) | 0.0 |
| 142820 Emphysema Lung 9 (Ardais) | 2.0 | 162593 Crohn's 47751 (NDRI) | 0.0 |
| 142821 Emphysema Lung 10 | 1.1 | | |
| (Ardais) | | | |

TABLE 12
Panel 3D
Column A - Rel. Exp.(%) Ag4347, Run 190952231

| Tissue Name | A | Tissue Name | A |
|---|---|---|---|
| 94905 Daoy | 0.0 | 94954 Ca Ski Cervical epidermoid | 0.0 |
| Medulloblastoma/Cerebellum | | carcinoma (metastasis | |
| 94906 TE671 | 0.0 | 94955 ES-2 Ovarian clear cell carcinoma | 0.0 |
| Medulloblastom/Cerebellum | | 94957 Ramos Stimulated with | 0.0 |
| 94907 D283 Med | 7.7 | PMA/ionomycin 6 h | |
| Medulloblastoma/Cerebellum | | 94958 Ramos Stimulated with | 0.0 |
| 94908 PFSK-1 Primitive | 3.7 | PMA/ionomycin 14 h | |
| Neuroectodermal/Cerebellum | | 94962 MEG-01 Chronic myelogenous | 0.0 |
| 94909 XF-498 CNS | 0.0 | leukemia (megokaryoblast) | |
| 94910 SNB-78 CNS/glioma | 27.9 | 94963 Raji Burkitt's lymphoma | 7.1 |
| 94911 SF-268 CNS/glioblastoma | 0.0 | 94964 Daudi Burkitt's lymphoma | 5.7 |
| 94912 T98G Glioblastoma | 0.0 | 94965 U266 B-cell | 0.0 |
| 96776 SK-N-SH Neuroblastoma | 3.5 | plasmacytoma/myeloma | |
| (metastasis) | | 94968 CA46 Burkitt's lymphoma | 6.7 |
| 94913 SF-295 CNS/glioblastoma | 19.3 | 94970 RL non-Hodgkin's B-cell | 0.0 |
| 94914 Cerebellum | 15.1 | lymphoma | |
| 96777 Cerebellum | 14.9 | 94972 JM1 pre-B-cell | 0.0 |
| 94916 NCI-H292 Mucoepidermoid lung | 14.1 | lymphoma/leukemia | |
| carcinoma | | 94973 Jurkat T cell leukemia | 0.0 |
| 94917 DMS-114 Small cell lung cancer | 63.7 | 94974 TF-1 Erythroleukemia | 0.0 |
| 94918 DMS-79 Small cell lung | 100.0 | 94975 HUT 78 T-cell lymphoma | 0.0 |
| cancer/neuroendocrine | | 94977 U937 Histiocytic lymphoma | 0.0 |
| 94919 NCI-H146 Small cell lung | 7.4 | 94980 KU-812 Myelogenous leukemia | 14.0 |
| cancer/neuroendocrine | | 769-P-Clear cell renal carcinoma | 13.8 |
| 94920 NCI-H526 Small cell lung | 1.2 | 94983 Caki-2 Clear cell renal carcinoma | 0.0 |
| cancer/neuroendocrine | | 94984 SW 839 Clear cell renal carcinoma | 19.6 |
| 94921 NCI-N417 Small cell lung | 0.0 | 94986 G401 Wilms' tumor | 7.8 |
| cancer/neuroendocrine | | 94987 Hs766T Pancreatic carcinoma (LN | 6.8 |
| 94923 NCI-H82 Small cell lung | 0.0 | metastasis) | |
| cancer/neuroendocrine | | 94988 CAPAN-1 Pancreatic | 0.0 |
| 94924 NCI-H157 Squamous cell lung | 0.0 | adenocarcinoma (liver metastasis) | |
| cancer (metastasis) | | 94989 SU86.86 Pancreatic carcinoma | 0.0 |
| 94925 NCI-H1155 Large cell lung | 4.5 | (liver metastasis) | |
| cancer/neuroendocrine | | 94990 BxPC-3 Pancreatic | 0.0 |
| 94926 NCI-H1299 Large cell lung | 0.0 | adenocarcinoma | |
| cancer/neuroendocrine | | 94991 HPAC Pancreatic adenocarcinoma | 0.0 |
| 94927 NCI-H727 Lung carcinoid | 0.0 | 94992 MIA PaCa-2 Pancreatic carcinoma | 0.0 |
| 94928 NCI-UMC-11 Lung carcinoid | 0.0 | 94993 CFPAC-1 Pancreatic ductal | 20.4 |
| 94929 LX-1 Small cell lung cancer | 0.0 | adenocarcinoma | |
| 94930 Colo-205 Colon cancer | 0.0 | 94994 PANC-1 Pancreatic epithelioid | 0.0 |
| 94931 KM12 Colon cancer | 0.0 | ductal carcinoma | |
| 94932 KM20L2 Colon cancer | 0.0 | 94996 T24 Bladder carcinma (transitional | 19.9 |
| 94933 NCI-H716 Colon cancer | 28.3 | cell | |
| 94935 SW-48 Colon adenocarcinoma | 7.2 | 5637-Bladder carcinoma | 0.0 |
| 94936 SW1116 Colon adenocarcinoma | 11.0 | 94998 HT-1197 Bladder carcinoma | 0.0 |
| 94937 LS 174T Colon adenocarcinoma | 13.5 | 94999 UM-UC-3 Bladder carcinma | 0.0 |
| 94938 SW-948 Colon adenocarcinoma | 0.0 | (transitional cell) | |
| 94939 SW-480 Colon adenocarcinoma | 0.0 | 95000 A204 Rhabdomyosarcoma | 14.4 |
| 94940 NCI-SNU-5 Gastric carcinoma | 10.5 | 95001 HT-1080 Fibrosarcoma | 0.0 |
| KATO III-Gastric carcinoma | 29.1 | 95002 MG-63 Osteosarcoma (bone) | 0.0 |
| 94943 NCI-SNU-16 Gastric carcinoma | 0.0 | 95003 SK-LMS-1 Leiomyosarcoma | 8.2 |
| 94944 NCI-SNU-1 Gastric carcinoma | 7.5 | (vulva) | |
| 94946 RF-1 Gastric adenocarcinoma | 0.0 | 95004 SJRH30 Rhabdomyosarcoma (met | 0.0 |
| 94947 RF-48 Gastric adenocarcinoma | 0.0 | to bone marrow) | |
| 96778 MKN-45 Gastric carcinoma | 23.2 | 95005 A431 Epidermoid carcinoma | 0.0 |
| 94949 NCI-N87 Gastric carcinoma | 0.0 | 95007 WM266-4 Melanoma | 5.2 |
| 94951 OVCAR-5 Ovarian carcinoma | 0.0 | DU 145-Prostate carcinoma (brain | 0.0 |
| 94952 RL95-2 Uterine carcinoma | 0.0 | metastasis) | |
| 94953 HelaS3 Cervical adenocarcinoma | 0.0 | 95012 MDA-MB-468 Breast | 0.0 |
| | | adenocarcinoma | |
| SCC-4-Squamous cell carcinoma of | 0.0 | | |
| | | tongue | |
| SCC-9-Squamous cell carcinoma of | 0.0 | | |
| | | tongue | |
| SCC-15-Squamous cell carcinoma of | 0.0 | | |
| | | tongue | |
| | | 95017 CAL 27 Squamous cell carcinoma | 6.7 |
| | | of tongue | |

TABLE 13
Panel 4.1D
Column A - Rel. Exp.(%) Ag4346, Run 190944493

| Tissue Name | A | Tissue Name | A |
|---|---|---|---|
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL-1beta | 0.0 |
| Primary Th2 act | 0.0 | Microvascular Dermal EC none | 0.0 |
| Primary Tr1 act | 0.0 | Microsvasular Dermal EC TNF alpha + IL-1beta | 0.0 |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL-1beta | 0.0 |
| CD45RA CD4 lymphocyte act | 0.0 | Coronery artery SMC rest | 0.0 |
| CD45RO CD4 lymphocyte act | 0.0 | Coronery artery SMC TNF alpha + IL-1beta | 0.0 |
| CD8 lymphocyte act | 0.0 | Astrocytes rest | 0.9 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1 anti-CD95 CH11 | 3.3 | CCD1106 (Keratinocytes) none | 0.7 |
| LAK cells rest | 0.7 | CCD1106 (Keratinocytes) TNF alpha + IL-1beta | 0.0 |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.5 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 2.3 | Lung fibroblast none | 0.0 |
| PBMC rest | 0.0 | Lung fibroblast TNF alpha + IL-1beta | 0.0 |
| PBMC PWM | 1.3 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and IL-4 | 0.0 | Dermal fibroblast CCD1070 TNF alpha | 0.0 |
| EOL-1 dbcAMP | 0.0 | Dermal fibroblast CCD1070 IL-1beta | 0.6 |
| EOL-1 dbcAMP PMA/ionomycin | 0.0 | Dermal fibroblast IFN gamma | 0.0 |
| Dendritic cells none | 0.0 | Dermal fibroblast IL-4 | 0.6 |
| Dendritic cells LPS | 0.0 | Dermal Fibroblasts rest | 0.0 |
| Dendritic cells anti-CD40 | 0.0 | Neutrophils TNFa + LPS | 1.1 |
| Monocytes rest | 0.0 | Neutrophils rest | 0.6 |
| Monocytes LPS | 0.0 | Colon | 0.5 |
| Macrophages rest | 0.0 | Lung | 2.2 |
| Macrophages LPS | 0.0 | Thymus | 12.2 |
| HUVEC none | 0.0 | Kidney | 100.0 |
| HUVEC starved | 1.3 | | |

TABLE 14
general oncology screening panel_v_2.4
Column A - Rel. Exp.(%) Ag4347, Run 260280470

| Tissue Name | A | Tissue Name | A |
|---|---|---|---|
| Colon cancer 1 | 58.6 | Bladder NAT 2 | 0.0 |
| Colon NAT 1 | 0.0 | Bladder NAT 3 | 0.0 |
| Colon cancer 2 | 0.0 | Bladder NAT 4 | 25.0 |
| Colon NAT 2 | 0.0 | Prostate adenocarcinoma 1 | 23.2 |
| Colon cancer 3 | 0.0 | Prostate adenocarcinoma 2 | 0.0 |
| Colon NAT 3 | 29.1 | Prostate adenocarcinoma 3 | 44.8 |
| Colon malignant cancer 4 | 21.8 | Prostate adenocarcinoma 4 | 0.0 |
| Colon NAT 4 | 0.0 | Prostate NAT 5 | 25.5 |
| Lung cancer 1 | 0.0 | Prostate adenocarcinoma 6 | 10.1 |
| Lung NAT 1 | 0.0 | Prostate adenocarcinoma 7 | 0.0 |
| Lung cancer 2 | 0.0 | Prostate adenocarcinoma 8 | 0.0 |
| Lung NAT 2 | 0.0 | Prostate adenocarcinoma 9 | 10.6 |
| Squamous cell carcinoma 3 | 0.0 | Prostate NAT 10 | 0.0 |
| Lung NAT 3 | 0.0 | Kidney cancer 1 | 24.7 |
| Metastatic melanoma 1 | 79.0 | Kidney NAT 1 | 24.3 |
| Melanoma 2 | 69.7 | Kidney cancer 2 | 75.3 |
| Melanoma 3 | 19.5 | Kidney NAT 2 | 28.1 |
| Metastatic melanoma 4 | 100.0 | Kidney cancer 3 | 7.4 |
| Metastatic melanoma 5 | 49.3 | Kidney NAT 3 | 23.7 |
| Bladder cancer 1 | 0.0 | Kidney cancer 4 | 0.0 |
| Bladder NAT 1 | 0.0 | Kidney NAT 4 | 28.1 |
| Bladder cancer 2 | 0.0 | | |

AI_comprehensive panel_v1.0 Summary: Ag4347 Highest expression of this gene was detected in normal lung (CT=30.8). This gene showed a wide spread low expression in this panel. Moderate to low levels of expression of this gene were detected in samples derived from normal and orthoarthitis/rheumatoid arthritis bone, cartilage, and synovium samples, from normal lung, COPD lung, emphysema, atopic asthma, asthma, allergy, Crohn's disease (normal matched control and diseased), ulcerative colitis (normal matched control and diseased), and psoriasis (normal matched control and diseased). For example, CG54455 is down-regulated in psoriasis patients and is up-regulated in its corresponding matched controls. Thus, gene or protein levels of expression of CG54455 are useful as a diagnostic marker for psoriasis. Furthermore, therapeutic modulation of CG54455, encoded protein and/or use of antibodies or small molecule targeting this gene or gene product is useful in the treatment of autoimmune and inflammatory disorders including psoriasis, allergy, asthma, inflammatory bowel disease, rheumatoid arthritis and osteoarthritis.

Cellular OA/RA Summary: Ag4346 Highest expression of this gene was detected in MH7A (synoviocyte) cell line (CT=33.5). Low expression of this gene was also detected in untreated and activated SW1353 (chondrocyte) cell lines, and activated MH7A cells. Decreased expression of CG54455 was detected in a cluster of samples derived from MH7A synoviocyte cells treated with anti-inflammatory compounds such as IL1b and TNFα. Thus, gene or protein levels of expression of CG54455 are useful to differentiate between these samples and other samples on this panel and as a marker to detect the presence of inflammatory disorders. Furthermore, modulation of this gene or encoded protein will be useful in the treatment of orthoarthritis and rheumatoid arthritis.

General_screening_panel_v1.4 Summary: Ag4347 Highest expression of this gene was detected in ovarian cancer SK-OV-3 cell line (CT=32). Low expression of this gene was detected in number of cancer cell lines derived from ovarian, breast, and colon cancers. Therefore, therapeutic modulaion of this gene, expressed protein and/or use of antibodies or small molecule drug targeting this gene or gene product is useful in the treatment of ovarian, breast and colon cancers.

Low levels of expression of this gene were seen in all the regions of the central nervous system examined, including amygdala, hippocampus, substantia nigra, thalamus, cerebellum, cerebral cortex, and spinal cord. Expression analysis of this gene using CuraChip 1.2 (see example 2) showed that this gene was down-regulated in the temporal cortex of Alzheimers patient but was up-regulated in patients who were found to have serious Alzheimer disease-like pathology with no associated dementia relative to the control patients. Therefore, therapeutic modulation of this gene, expressed protein and/or use of antibodies or small molecule drug targeting this gene or gene product is useful in the treatment of central nervous system disorders such as Alzheimers disease, Parkinson's disease, epilepsy, multiple sclerosis, schizophrenia and depression.

Mini tissue OA/RA Summary: Ag4346 Highest expression of this gene was detected in brain (CT=29.9). Significant expression of this gene was also seen in normal synovium, kidney, colon, ovary and small intestine. Expression of this gene was slightly downregulated in synovium from OA and RA patents. Therefore, modulation of this gene and/or encoded protein is useful in the treatment of orthoarthritis and rheumatoid arthritis.

PGI1.0 Summary: Ag4346 Highest expression of this gene was detected in lung fibrosis sample (CT=30.8). Significant levels of expression of this gene was also detected in emphyzema and asthma lung. Therefore, therapeutic modulaion of this gene, encoded protein and/or use of expressed protein, antibodies or small molecule drug targeting this gene or gene product is useful in the treatment of emphyzema, asthma and lung fibrosis.

Panel 3D Summary: Ag4347 Low expression of this gene was detected mainly in a small cell lung cancer DMS-79 and DMS-114 cell lines (CTs=33-34). Therefore, therapeutic modulation of this gene, encoded protein, and/or use of small molecule drug targeting this gene or gene product is useful in the treatment of small cell lung cancer.

Panel 4.1D Summary: Ag4346 Moderate levels of expression of this gene was detected mainly in kidney sample (CT=31.8). Therefore, therapeutic modulation of this gene is useful in the treatment of kidney related diseases including lupus erythematosus and glomerulonephritis.

General oncology screening panel_v_{—}2. 4 Summary: Ag4347 Low expression of this gene was detected in a colon cancer, a metastatic melanoma and a kidney cancer samples. Therefore, therapeutic modulation of this gene, encoded protein, and/or use of antibodies or small molecule drug targeting this gene or gene product is useful in the treatment of these cancers.

### 6.16. Example 16

### Expression Analysis

A new panel consisting of human tissues of normal skin, kelloid tissue, stomach, small intestine, lung and brain was generated to investigate the expression of CG54455 in these tissues. The Epiderm skin model (EPI-200) from Mattek Corporation was also used. This system consists of normal human-derived epidermal keratinocytes which have been cultured to form a multilayered, highly differentiated model of the human epidermis. EPI-200 cultures were collected at different time point of the culture, day 0 (completely undifferentiated), day 2, 3, 4, 5 and 7 (tissue differentiated expressing K1 and K10 (information provided by the manufacturer). These samples are referred as EPI-200 0, 2A, 3A, 4A, 5A, 7A. Undifferentiated culture obtained at day 3, was also collected and named 3S. Epi-200 fully differentiated (day 14) was also treated either with PMA or UVA 11.5J or solar UV for 6 hours. Treated and non-treated samples were then processed for RNA isolation.

RNA was isolated from these tissues either in house or purchased.

The quantitative tissue expression of CG54455 was assessed by real time quantitative PCR (TAQMAN®) performed on a Perkin-Elmer Biosystems ABI PRISM® 7700 Sequence Detection System (See Section 6.15). Briefly, RNA was converted to cDNA and analyzed via TAQMAN® using One Step RT. PCR Master Mix Reagents (PE Biosystems; cat. # 4309169) and gene-specific primers according to the manufacturer's instructions. cDNA was then normalized to β-actin, GADPH, ADPR. Probes and primers were designed for CG54455 to Perkin Elmer Biosystem's Primer Express Software package (version I for Apple Computer's Macintosh Power PC) using the nucleic acid sequences of the invention as input. Probes and primers used for each gene are listed below:

TABLE 15
Probe/Primers for Ag7129

| | | | |
|---|---|---|---|
| | | | Start |
| Primers | Sequences | Length | Position |

| | | | |
|---|---|---|---|
| Forward | 5′-gtggaaaagaacggcagtaaata-3′ | 23 | 936 |
| | (SEQ ID NO: 53) | | |
| Probe | TET-5′-tgccctacctcaaggttctcaagcac-3′-TAMRA | 26 | 973 |
| | (SEQ ID NO: 54) | | |
| Reverse | 5′-acttctgcattggaactatttatcc-3′ | 25 | 1003 |
| | (SEQ ID NO: 55) | | |

TABLE 16
Probe Name: Ag5888

| | | | | |
|---|---|---|---|---|
| | | | | Start |
| Primers | Sequences | TM | Length | Position |

| | | | | |
|---|---|---|---|---|
| Forward | 5′-CAGTAGTTTTCCAGCCTTTCTTG-3′ | 59.5 | 23 | 43 |
| | (SEQ ID NO: 56) | | | |
| Probe | FAM-5′-TGGACTGTTTTCTTTCTTCTCAAAATTTTC-3′-TAMRA | 64.8 | 30 | 88 |
| | (SEQ ID NO: 57) | | | |
| Reverse | 5′-TCCAGCAGGGAGATTTCTTT-3′ | 58.9 | 20 | 121 |
| | (SEQ ID NO: 58) | | | |

TABLE 17
Probe/Primers for Ag6824

| | | | |
|---|---|---|---|
| Primers | Sequences | Length | Start Position |

| | | | |
|---|---|---|---|
| Forward | 5′-gaggccaccaactcttcttc-3′ | 20 | 160 |
| | (SEQ ID NO: 59) | | |
| Probe | TET-5′-ctcctccttctcctctccttccagc-3′-TAMRA | 25 | 180 |
| | (SEQ ID NO: 60) | | |
| Reverse | 5′-gtgattgtagctccgcacat-3′ | 20 | 215 |
| | (SEQ ID NO: 61) | | |

PCR cocktails including Probes and primers were set up using 1× TaqMan™ PCR Master Mix for the PE Biosystems 7700, with 5 mM MgCl₂, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Results were recorded as CT values (cycle at which a given sample crosses a threshold level of fluorescence) using a log scale, with the difference between two samples being represented as 2 to the power of delta CT.

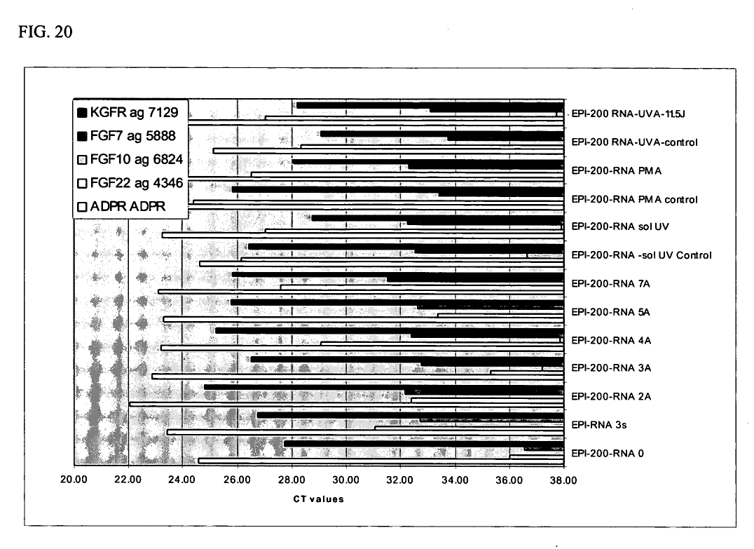

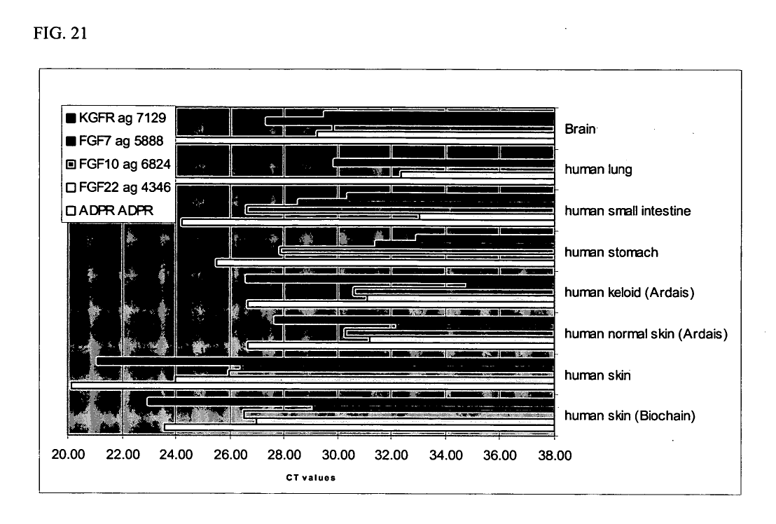

A summary of the expression results is presented in FIGS. 20 and 21. Expression in the indicated tissue is presented as CT values.

CG54455 was found expressed at the highest level in EPI-200 cultured for 7 days and fully differentiated EP-200 (day 14) (CT values 26-27). No expression was found in complete undifferentiated keratinocytes, EPI-200 0.

Moderate to high expression of CG54455 was also found in normal skin tissues (CT 24 to 31). These expression data indicate that CG54455 is expressed in human skin and particular in the epidermis. To the contrary, FGF7 and FGF 10 are not expressed or at very low level in the epidermis

In summary, the data strongly suggest that CG54455 is a novel member of the KGF family. Its expression differs from the two other members of the family, FGF7 and FGF10, CG54455 being an epithelial KGF, and FGF7 and FGF10 being mesenchymal KGFs.

### 6.17. Example 17

### Polyclonal Antibodies Against CG54455-14

Peptides were designed and selected as immunogen for the generation of polyclonal antibodies against CG54455 as shown in Table 18. Peptide synthesis, conjugation, immunization were outsourced to Rockland Immunochemicals (quote 2901) and New England Peptide.

TABLE 18
Peptide generated for use as Immunogen

| | | |
|---|---|---|
| Peptide | Rabbit(s) | Outsourcing Company |

| | | |
|---|---|---|
| H2N-CPGGRTRRYHLS-OH | 2 | New England Peptide |
| (SEQ ID NO: 48) | | |
| H2N-CSQRWRRRGQP-OH | 2 | New England Peptide |
| (SEQ ID NO: 49) | | |
| Ac-CSTHFFLRVDPGGRVQ-amide | 2 | Rockland Immunochemicals |
| (SEQ ID NO: 50) | | |
| Ac-CRFRERIEENGHN-amide | 2 | Rockland Immunochemicals |
| (SEQ ID NO: 51) | | |
| Ac-CRPGGRTRRYHLS-amide | 2 | Rockland Iimmunochemicals |
| (SEQ ID NO: 52) | | |

Antibody Generation by New England Peptide:

Two Rabbits were immunized for each peptides. In total, 4 rabbits were immunized. Screening of the antibodies was done in house by ELISA and Western blot analysis.

ELISA: CG54455-06, CG54455-14, FGF7 and FGF10 (R&D systems), CG53135 (FGF-20) and unrelated proteins (CG90709-08-1 and albumin) were used as antigens. The 96 well ELISA plates were coated with the antigen and subjected to standard ELISA protocol. Binding of the polyclonal antibodies against CG54455 was revealed by the addition of an anti-rabbit IgG antibody conjugated to HRP (Amersham) and detected after the addition of the TMB substrate (PharMingen). Reaction was monitored by reading of the absorbance at 450 nm.

Western blot: CG54455-06, CG54455-14 and unrelated protein (mouse serum albumin) were used as antigens. The antigen-antibody binding reaction was detected using anti-rabbit IgG antibody conjugated to HRP and the ECL kit (Amersham).

After screening, one polyclonal antibody (B4980) was selected based on its strong specificity for CG54455 for further study, for example, as detection tool for purification of CG54455-14. B4980 polyclonal antibodies showed strong specificity for CG54455-06, however, some cross reactivity to V5-His-tagged protein was observed. Therefore, other polyclonal antibodies were generated.

Antibody Generation by Rockland Immunochemicals:

Two Rabbits were immunized with each KLH-conjugated peptides or a pool of the 3 KLH-conjugated peptides. In total 8 rabbits were immunized. Screening was done in house by ELISA and Western blot analysis as described in the above section. After screening one polyclonal antibody was selected based on its strong specificity for CG54455 for further study. This antibody was referred to as PAB-404-882A.

### 6.18. Example 18

### Monoclonal Antibodies

CG54455-06 was used as immunogen for the generation of mouse monoclonal antibodies. The generation of these antibodies were outsourced to Rockland Immunochemicals and was done according their standard procedure. The screening was done in house using the same scheme as for the polyclonal antibodies (see above).

Two monoclonal antibodies were selected based on their specificity by ELISA and Western blot analysis (5F7G8 and 5F7H5).

The data strongly suggest that CG54455 is a novel member of the KGF family. Its expression differs from the two other members of the family, FGF7 and FGF10, CG54455 being an epithelial KGF, and FGF7 and FGF10 being mesenchymal KGFs.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

What is claimed is:

1. An isolated nucleic add molecule selected from the group consisting of:
(a) a nucleic acid molecule comprising a nucleotide sequence selected from the group consisting of SEQ ID NOs: 3, 5, 6, 16, 18, 29, nucleotides 7-453 of SEQ ID NO:9, nucleotides 12-524 of SEQ ID NO: 10, nucleotides 17-518 of SEQ ID NO: 11, nucleotides 12-530 of SEQ ID NO: 12, nucleotides 11-457 of SEQ ID NO: 14, nucleotides 4-453 of SEQ ID NO: 20, nucleotides 7-456 of SEQ ID NO: 22, nucleotides 8-457 of SEQ ID NO: 23, nucleotides 8-446 of SEQ ID NO: 25, and nucleodies 8-446 of SEQ ID NO: 27;
(b) a nucleic acid molecule encoding a protein comprising an amino acid sequence selected from the group consisting of SEQ ID NOs: 2, 4, 7, 13, 15, 17, 19, 21, 24, 26 28, and 30;
(c) a nucleic acid molecule hybridizes under stringent conditions to a nucleotide sequence of SEQ ID NO: 5 or nucleotides 7-456 of SEQ ID NO: 22, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA;
(d) a fragment of an nucleic acid molecule of any of (a)-(c); and
(e) a complement of an nucleic acid molecule of any of (a)-(d).

2. The isolated nucleic acid molecule of claim 1 comprising SEQ ID NO:5.

3. The isolated nucleic acid molecule of claim 1 comprising nucleotides 7-456 of SEQ ID NO:22.

4. A vector comprising the nucleic acid molecule of claim 1.

5. The vector of claim 4, wherein said nucleic acid molecule is operably linked to an expression control sequence.

6. A prokaryotic or eukaryotic host cell containing a nucleic acid molecule of claim 1.

7. A prokaryotic or eukaryotic host cell containing the vector of claim 4.

8. A prokaryotic or eukaryotic host cell containing the vector of claim 5.

9. A method comprising culturing the host cell of claim 7 or 8 in a suitable nutrient medium.

10. The method of claim 9, wherein said host cell is E. coli.

11. The method of claim 9 further comprising isolating a protein encoded by said nucleic acid molecule from said cultured cells or said nutrient medium.

12. An isolated protein by method of claim 11.

13. An isolated protein selected from the group consisting of:
(a) a protein comprising an amino acid sequence of SEQ ID NO: 2, 4, 7, 13, 15, 17, 19, 21, 24, 26, 28 or 30;
(b) a protein with one or more amino acid substitutions to the protein of (a), wherein said substitutions are no more than 15% of the amino acid sequence of SEQ ID NO: 2, 4, 7, 13, 15, 17, 19, 21, 24, 26, 28 or 30, and wherein said protein with one or more amino acid substitutions retains cell proliferation stimulatory activity; and
(c) a fragment of the protein of (a) or (b).

14. The isolated protein of claim 13 comprising an amino acid sequence of SEQ ID NO:2.

15. The isolated protein of claim 14 comprising an amino acid sequence of SEQ ID NO:21.

16. An isolated polypeptide comprising an amino acid sequence, wherein said amino acid sequence has one or more conservative amino acid substitutions relative to SEQ ID NO: 2, 4, 7, 13, 15, 17, 19, 21, 24, 26, 28 or 30.

17. An isolated polypeptide comprising an amino acid sequence, wherein said amino acid sequence is a fragment of SEQ ID NO: 2, 4, 7, 13, 15, 17, 19, 21, 24, 26, 28 or 30, and wherein said fragment retains cellular proliferation stimulatory activity.

18. A pharmaceutical composition comprising a pharmaceutically acceptable carrier, and an isolated protein of claim 13.

19. A method of preventing or treating a disorder associated with pathology of epithelial cells comprising administering to a subject in need thereof an effective amount of a composition comprising an isolated protein of claim 13.

20. A method of stimulating proliferation, differentiation or migration of epithelial cells comprising administering to a subject in need thereof an effective amount of a composition comprising an isolated protein of claim 13.

21. The method of claim 19 or 20, wherein said composition further comprising a pharmaceutically acceptable carrier.

22. The method of any of claims 19 or 20, wherein said epithelial cells locate at the alimentary tract of said subject.

23. The method of claim 19 or 20, wherein said epithelial cells locate at the pulmonary tract of said subject.

24. The method of any of claims 19 or 20, wherein said subject is a mammal.

25. The method of claim 24, wherein said mammal is a human.
